(19) United States
(12) Patent Application Publication
(10) Pub. No.: US 2019/0358808 A1
(43) Pub. Date: Nov. 28, 2019

(54) ASSIST DEVICE

(71) Applicant: JTEKT CORPORATION, Osaka-shi (JP)

(72) Inventors: Yoshitaka YOSHIMI, Kashiba-shi (JP); Toshiki KUMENO, Kyoto-shi (JP)

(73) Assignee: JTEKT CORPORATION, Osaka-shi (JP)

(21) Appl. No.: 16/422,287

(22) Filed: May 24, 2019

(30) Foreign Application Priority Data
May 28, 2018 (JP) 2018-101839
May 28, 2018 (JP) 2018-101840

(51) Int. Cl. B25J 9/00; A61H 3/00; A61B 5/00; A61H 1/02

(52) U.S. Cl. CPC B25J 9/0006; A61H 3/008; A61B 5/6831; A61H 2201/1628; A61H 2201/165; A61H 2201/5061; A61H 2201/5069; A61H 1/0244

(57) ABSTRACT

An assist device includes: body gear worn at least around hips of a person being assisted; an actuator unit attached to the body gear and worn on thighs of the person being assisted; an angle detection part configured to detect a forward leaning angle of the hips relative to the thighs of the person being assisted; and a controller configured to control the actuator unit. The actuator unit is configured to generate an assisting torque that assists the person in moving his or her thighs relative to the hips or moving his or her hips relative to the thighs. The controller is configured to execute at least one of first control and second control.

### INCORPORATION BY REFERENCE

The disclosure of Japanese Patent Application No. 2018-101839 filed on May 28, 2018 and Japanese Patent Application No. 2018-101840 filed on May 28, 2018, each including the specification, drawings and abstract, is incorporated herein by reference in its entirety.

### BACKGROUND

### 1. Technical Field

The present disclosure relates to an assist device that assists a person being assisted in moving his or her body parts to be assisted.

### 2. Description of Related Art

For example, Japanese Patent Application Publication No. 2013-173190 (JP 2013-173190 A) describes a wearable motion assist device that assists a person being assisted in moving his or her thighs relative to the hips when lifting a heavy object by bending down and straightening up from the hips or when walking normally. This wearable motion assist device includes a hip frame worn on the hips of the person being assisted, a back support, a belly support, coupling members coupling together the back support and the belly support, thigh-fixed parts fixed to the thighs, and a driving mechanism that drives the thigh-fixed parts relative to the hip frame. The wearable motion assist device further includes biological signal detection sensors attached to the skin of the person being assisted, and a control unit that controls the driving mechanism based on biological signals output from the biological signal detection sensors. To detect from the skin bioelectrical potential signals, such as myoelectric potential signals and neurotransmission signals, the biological signal detection sensor has an electrode that detects a weak electrical potential. The biological signal detection sensors are attached to the skin of the person being assisted, on the front sides of the right and left thighs near the hips, on the inner sides of the right and left thighs near the hips, on the right and left buttocks, in right- and left-side areas, a little above the hips, of the back, etc., by means of an adhesive sticker covering a periphery of the electrode.

### SUMMARY

The wearable motion assist device described in JP 2013-173190 A requires many biological signal detection sensors, which need to be attached to numerous parts of the person being assisted, including the front sides of the right and left thighs, the inner sides of the right and left thighs, the right and left buttocks, and the right- and left-side areas of the back. This makes it extremely troublesome for a person being assisted to wear the wearable motion assist device to use the device. Before attaching the biological signal detection sensors, the positions and the number of sensors to be attached (e.g., attaching three sensors close to one another at each measurement point) need to be determined, which is also troublesome. Moreover, the process of removing noise from the weak biological signals output from the many biological signal detection sensors, and the process of providing assistance by inferring what kind of motion is being performed (lifting a heavy object, walking, etc.) based on the biological signals output from the biological signal detection sensors, can become extremely complicated.

In particular, when assisting a person being assisted in performing a motion of lifting up a load or a motion of lowering (putting down) a load, if the motion of a body part to be assisted of the person being assisted is a slow motion, the wearable motion assist device described in JP 2013-173190 A may experience a delay in the process of inferring what kind of motion is being performed and fail to provide a sufficient assisting torque.

The present disclosure can be easily worn by a person being assisted, and can more appropriately assist the person being assisted in performing a load lifting motion or a load lowering motion while using a simpler configuration and simpler control.

A first aspect of the present disclosure is an assist device. This assist device includes: body gear worn at least around hips of a person being assisted; an actuator unit attached to the body gear and worn on thighs of the person; an angle detection part configured to detect a forward leaning angle of the hips relative to the thighs of the person; and a controller configured to control the actuator unit. The actuator unit is configured to generate an assisting torque that assists the person in moving his or her thighs relative to the hips or moving his or her hips relative to the thighs. The controller is configured to execute at least one of first control and second control. The first control involves, during a lifting task, obtaining a lifting assisting torque based on the forward leaning angle detected by using the angle detection part, an angular velocity-related amount based on a change in the forward leaning angle, and a reference lifting characteristic, and then driving the actuator unit based on the assisting torque that is the lifting assisting torque. The lifting task is a task in which the person lifts a load that the person is holding in a forward leaning posture, while gradually reducing the forward leaning angle. The second control involves, during a lowering task, obtaining a lowering assisting torque that is a torque in a lifting direction, based on the forward leaning angle detected by using the angle detection part and the angular velocity-related amount based on the change in the forward leaning angle, and then driving the actuator unit based on the assisting torque that is the obtained lowering assisting torque. The lowering task is a task in which the person lowers a load that the person is holding, in a lowering direction opposite from the lifting direction while gradually increasing the forward leaning angle.

Compared with the wearable motion assist device described in JP 2013-173190 A, the assist device thus configured can be very easily worn by a person being assisted, who has only to wear the body gear at least around his or her hips and the actuator unit on his or her thighs and attach the actuator unit to the body gear. Since the lowering assisting torque in the lifting direction is obtained based on the forward leaning angle detected by using the angle detection part and the angular velocity-related amount based on a change in the forward leaning angle, both the structure and control of the assist device are simpler than those of the wearable motion assist device described in JP 2013-173190 A. Moreover, since the lifting assisting torque in the lifting direction is obtained based on the forward leaning angle detected by using the angle detection part, the angular velocity-related amount based on a change in the forward leaning angle, and the selected reference lifting characteristic, both the structure and control of the assist device are simpler than those of the wearable motion assist device described in JP 2013-173190 A.

The above assist device may further include a torque detection part configured to detect a person exerted torque change amount that is an amount of change in a person exerted torque that is a torque input from the person into the actuator unit as the person moves his or her thighs relative to the hips or moves his or her hips relative to the thighs by himself or herself. The controller may be configured to, when the person is performing a forward leaning motion of gradually increasing the forward leaning angle from an upright standing posture during the lowering task, detect the person exerted torque change amount at predetermined time intervals based on the angular velocity-related amount detected by using the torque detection part. The controller may be configured to obtain an amount of assistance according to the person exerted torque change amount, and to obtain the lowering assisting torque based on an integrated amount of assistance obtained by integrating the amount of assistance.

With the above configuration, the controller obtains the amount of assistance according to the person exerted torque change amount, and obtains the lowering assisting torque based on the integrated amount of assistance obtained by integrating the amount of assistance. Thus, simple control can be realized.

In the above assist device, the controller may be configured to, when a predetermined state arises during the lowering task, stop updating and retain the integrated amount of assistance, and then obtain the lowering assisting torque based on the retained integrated amount of assistance. The predetermined state may be either a state where the forward leaning angle has stopped changing as the person has stopped the forward leaning motion or a state where the person is performing an upright standing motion of gradually reducing the forward leaning angle from the forward leaning posture.

With the above configuration, the controller retains the lowering assisting torque in the lifting direction even when the person stops in the middle of a forward leaning motion during a lowering task in which the person holds a load in an upright standing posture and then puts down the load while gradually leaning forward. Thus, the assist device can appropriately provide assistance in the lowering task.

The above assist device may further include a storage unit. The storage unit may be configured to store a forward leaning angle-vs-lowering torque limit value characteristic having a torque limit value set according to the forward leaning angle. The controller may be configured to, during the lowering task, obtain the torque limit value based on the forward leaning angle and the forward leaning angle-vs-lowering torque limit value characteristic that is stored in the storage unit. The controller may be configured to use the integrated amount of assistance or the torque limit value, whichever is the smaller, as the lowering assisting torque.

With the above configuration, the controller has the torque limit value according to the forward leaning angle, and can thereby limit an excess lowering assisting torque and obtain an appropriate amount of lowering assisting torque according to the forward leaning angle.

The above assist device may further include a manipulation unit provided with at least one of a gain changing part and an amount increasing speed changing part. The gain changing part may be configured to allow the person to change a gain in the lowering assisting torque. The amount increasing speed changing part may be configured to allow the person to change a speed with which an amount of the lowering assisting torque is increased. The manipulation unit may be separate from the body gear and the actuator unit. When the manipulation unit is provided with the gain changing part, the controller may be configured to, during the lowering task, increase and decrease at least either a gain used to obtain the lowering assisting torque, or a torque limit value according to the forward leaning angle, based on an input from the person into the gain changing part. When the manipulation unit is provided with the amount increasing speed changing part, the controller may be configured to change the speed with which the amount of the lowering assisting torque is increased, based on an input from the person into the amount increasing speed changing part.

With the above configuration, the assist device has the manipulation unit provided with at least one of the gain changing part and the amount increasing speed changing part, which offers the convenience of adjusting at least one of the gain and the amount increasing speed according to the motion of the person during the lowering task.

The above assist device may further include a manipulation unit provided with a motion switching part. The motion switching part may be configured to switch between lowering assistance of assisting the person in performing a motion during the lowering task and lifting assistance of assisting the person in performing a motion during the lifting task. The manipulation unit may be separate from the body gear and the actuator unit. The controller may be configured to, when the motion switching part represents the lowering assistance during the lowering task, obtain the lowering assisting torque and then drive the actuator unit based on the assisting torque that is the lowering assisting torque.

With the above configuration, the assist device offers the convenience of switching between the lowering assistance and the lifting assistance by the manipulation unit. Since the lifting assistance and the lowering assistance are switched by the motion switching part, the assist device is unlikely to accidentally provide lifting assistance during a lowering task.

The above assist device may further include a storage unit. The storage unit may be configured to store a plurality of reference lifting characteristics having a set lifting assisting torque that is a torque in a lifting direction. The controller may be configured to, during the lifting task, select an applicable reference lifting characteristic from the reference lifting characteristics stored in the storage unit, and obtain the lifting assisting torque based on the forward leaning angle detected by using the angle detection part, the angular velocity-related amount based on the change in the forward leaning angle, and the selected reference lifting characteristic, and then drive the actuator unit based on the assisting torque that is the lifting assisting torque.

Compared with the wearable motion assist device described in JP 2013-173190 A, the assist device thus configured can be very easily worn by a person, who has only to wear the body gear at least around his or her hips and the actuator unit on his or her thighs and attach the actuator unit to the body gear. Since the lifting assisting torque in the lifting direction is obtained based on the forward leaning angle detected by using the angle detection part, the angular velocity-related amount based on a change in the forward leaning angle, and the selected reference lifting characteristic, both the structure and control of the assist device are simpler than those of the wearable motion assist device described in JP 2013-173190 A.

The above assist device may further include a torque detection part configured to detect a person exerted torque change amount that is an amount of change in a person exerted torque that is a torque input from the person into the actuator unit as the person moves his or her thighs relative to the hips or moves his or her hips relative to the thighs by himself or herself. Each of the reference lifting characteristics may have a plurality of motion states set according to a lifting state. The lifting state may include at least one of a virtual elapsed time based on a time that has elapsed since the person started to lift a load, the forward leaning angle, and the person exerted torque change amount. The controller may be configured to, during the lifting task, shift each of the motion states in the reference lifting characteristics based on the lifting state, and obtain the lifting assisting torque by a calculation method that is preset for each of the motion states.

With the above configuration, the controller has the plurality of motion states and shifts the motion state according to the lifting state. With the calculation method of the lifting assisting torque set for each motion state, the controller can obtain an appropriate lifting assisting torque according to the lifting state.

In the above assist device, the controller may be configured to, during the lifting task, when the reference lifting characteristic currently selected is different from the reference lifting characteristic selected last time or when the motion state has shifted to a predetermined motion state among the motion states, make an on-switching torque difference-reducing correction of reducing a predetermined difference. The predetermined difference may be a difference between the lifting assisting torque obtained based on the reference lifting characteristic selected last time and the lifting assisting torque obtained based on the reference lifting characteristic currently selected.

With the above configuration, the controller can allow the lifting assisting torque to change smoothly by appropriately preventing the lifting assisting torque from changing rapidly when the selected reference lifting characteristic has changed.

In the above assist device, in the predetermined motion state in each of the reference lifting characteristics, the lifting assisting torque may be set according to the virtual elapsed time. The controller may be configured to, during the lifting task, when the motion state has shifted to the predetermined motion state or when the reference lifting characteristic currently selected is different from the reference lifting characteristic selected last time, obtain a temporary lifting assisting torque based on a current virtual elapsed time and the reference lifting characteristic selected last time, and obtain a torque difference-reducing virtual elapsed time that is the virtual elapsed time corresponding to the temporary lifting assisting torque in the reference lifting characteristic currently selected, and then change the current virtual elapsed time to the torque difference-reducing virtual elapsed time.

With the above configuration, the controller can, in a relatively easy and simple manner, allow the lifting assisting torque to change smoothly by appropriately preventing the lifting assisting torque from changing rapidly when the selected reference lifting characteristic has changed.

### BRIEF DESCRIPTION OF THE DRAWINGS

Features, advantages, and technical and industrial significance of exemplary embodiments of the disclosure will be described below with reference to the accompanying drawings, in which like numerals denote like elements, and wherein:

FIG. 1 is a perspective view illustrating an example of the overall configuration of an assist device;

FIG. 2 is a perspective view illustrating an example of the overall configuration of an assist device in which a jacket is partially changed from that of the assist device of FIG. 1;

FIG. 3 is an exploded perspective view of the assist device shown in FIG. 1;

FIG. 4 is a perspective view illustrating an example of the external appearance of body gear of the assist device shown in FIG. 1;

FIG. 5 is a perspective view illustrating an example of the external appearance of an actuator unit of the assist device shown in FIG. 1;

FIG. 6 is a perspective view illustrating an example of the external appearance of a frame that is a component of the body gear;

FIG. 7 is a perspective view illustrating an example of the external appearance of a hip support that is a component of the body gear;

FIG. 8 is a development illustrating an example of the structure of the hip support;

FIG. 9 is a perspective view illustrating an example of the external appearance of a backpack (and a portion of the frame) that is a component of the body gear;

FIG. 10 is a perspective view illustrating an example of the external appearance of the jacket that is a component of the body gear, in a state of being connected to the backpack and the frame;

FIG. 11 is a development illustrating an example of the structure of the jacket;

FIG. 12 is a perspective view of a (right) actuator unit of the assist device shown in FIG. 1;

FIG. 13 is a perspective view illustrating another example of the (right) actuator unit shown in FIG. 12;

FIG. 14 is a perspective view illustrating the structure around a thigh-worn part (body holding part);

FIG. 15 is a view illustrating an example in which an under-knee belt is added to the body holding part shown in FIG. 14;

FIG. 16 is a view illustrating an example in which a third joint of the thigh-worn part (body holding part) of the (right) actuator unit shown in FIG. 13 is disposed on a front surface of the thigh of a person being assisted;

FIG. 17 is a view illustrating an example in which the third joint of the thigh-worn part (body holding part) of the (right) actuator unit shown in FIG. 13 is disposed on an outer lateral surface of the thigh of the person being assisted;

FIG. 18 is a view illustrating an example in which the third joint of the thigh-worn part (body holding part) of the (right) actuator unit shown in FIG. 13 is disposed on a back surface of the thigh of the person being assisted;

FIG. 19 is an exploded perspective view illustrating an example of the internal structure of the actuator unit;

FIG. 20 is a sectional view illustrating an example of the internal structure of the actuator unit;

FIG. 21 is a view illustrating an upright standing posture in which the person being assisted wearing the assist device stands with a straight back;

FIG. 22 is a view illustrating a state where the person being assisted has assumed a forward leaning posture from the posture shown in FIG. 21 and the frame etc. have turned around an imaginary turning axis;

FIG. 23 is a view illustrating an example of the external appearance of a manipulation unit;

FIG. 24 is a view illustrating inputs into and outputs from a controller;

FIG. 25 is tables illustrating changes (adjustments) made from the manipulation unit to a motion mode, a gain, and an amount increasing speed;

FIG. 26 is a control block diagram showing how the controller controls the actuator unit;

FIG. 27 is a flowchart illustrating an entire processing procedure based on the control block diagram shown in FIG. 26;

FIG. 28 is a flowchart illustrating details of a process [S100: adjustment determination, input processing, and torque change amount etc. calculation] in the flowchart shown in FIG. 27;

FIG. 29 is a flowchart illustrating details of a process [S200: motion type determination] in the flowchart shown in FIG. 27;

FIG. 30 is a flowchart illustrating details of a process [SD000R: (right) lowering] in the flowchart shown in FIG. 27;

FIG. 31 is a view illustrating how the person being assisted performs a lowering task;

FIG. 32 is a graph illustrating an example of a person being assisted-exerted torque change amount-vs-amount of assistance characteristic;

FIG. 33 is a graph illustrating an example of a forward leaning angle-vs-lowering torque limit value characteristic;

FIG. 34 is a graph illustrating how the forward leaning angle and the lowering assisting torque change over time while the person being assisted performs a lowering task;

FIG. 35 is a flowchart illustrating details of a process [SU000: lifting] in the flowchart shown in FIG. 27;

FIG. 36 is a state shift chart illustrating details of a process [SS000: motion state determination] in the flowchart shown in FIG. 35;

FIG. 37 is a graph illustrating how the forward leaning angle and the lifting assisting torque change as the motion state shifts while the person being assisted performs a lifting task;

FIG. 38 is a flowchart illustrating details of a process [SS100R: (right) amount increasing speed switching determination] in the flowchart shown in FIG. 35;

FIG. 39 is a graph illustrating examples of a time-vs-switching lower limit characteristic and a time-vs-switching upper limit characteristic;

FIG. 40 is a table illustrating an example of an amount increasing speed-vs-shift time characteristic;

FIG. 41 is a graph illustrating an example of a time-vs-amount of assistance characteristic;

FIG. 42 is a flowchart illustrating details of a process [SS170R: (right) assisting torque calculation] in the flowchart shown in FIG. 35;

FIG. 43 is graphs illustrating examples of a time-vs-lifting torque characteristic and a forward leaning angle-vs-maximum lifting torque characteristic;

FIG. 44 is a table illustrating an example of a gain-vs-damping coefficient characteristic; and

FIG. 45 is a graph illustrating an example of an assistance ratio-vs-torque damping ratio coefficient.

### DETAILED DESCRIPTION OF EMBODIMENTS

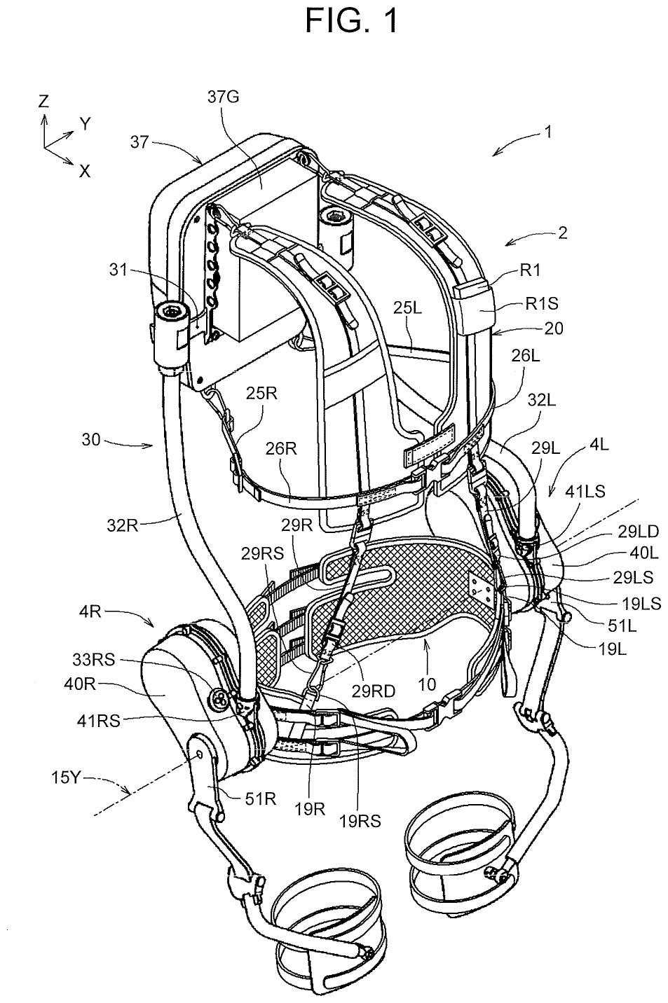

The overall structure of an assist device 1 will be described below based on FIG. 1 to FIG. 25. The assist device 1 is a device that assists a person being assisted, for example, in turning his or her thighs relative to the hips (or his or her hips relative to the thighs) when lifting a load (or lowering a load) and in turning his or her thighs relative to the hips when walking. The X-axis, Y-axis, and Z-axis in the drawings are orthogonal to one another, and as seen from the person being assisted wearing the assist device, an X-axis direction, Y-axis direction, and Z-axis direction correspond to a forward direction, leftward direction, and upward direction, respectively.

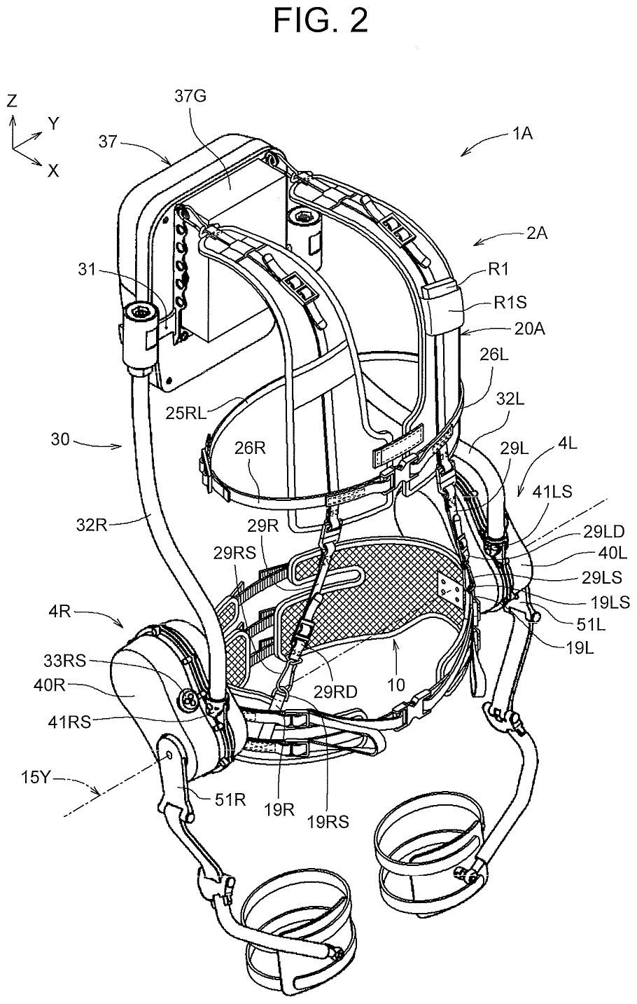

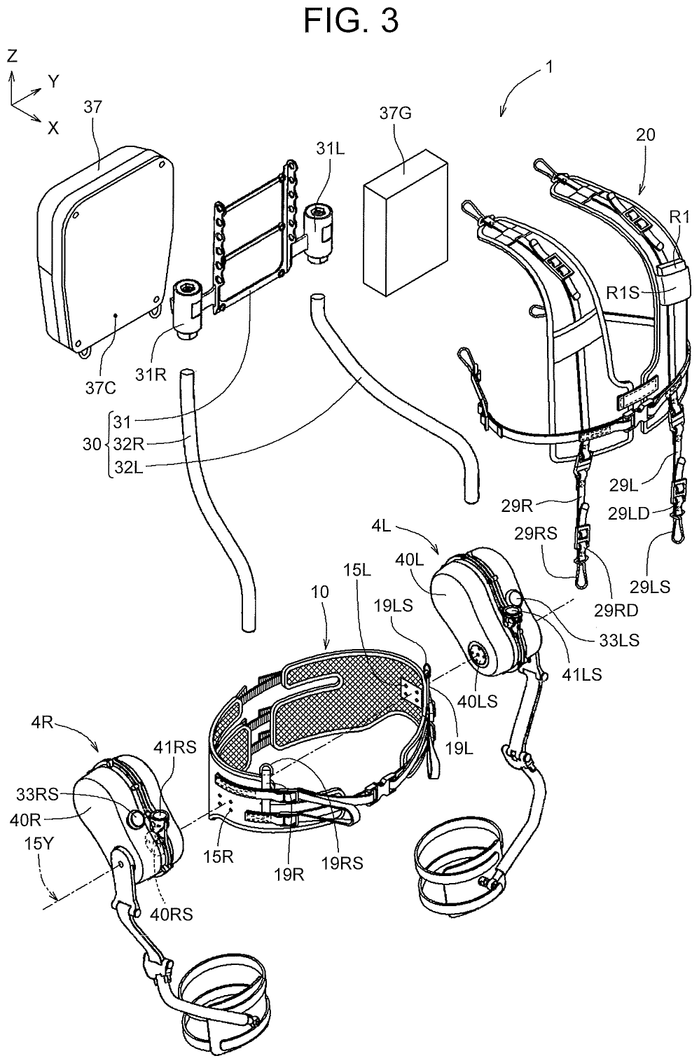

FIG. 1 shows the overall external appearance of the assist device 1. FIG. 2 shows the overall external appearance of an assist device 1A in which a close-fitting belt 25RL replaces a right side belt 25R and a left side belt 25L of FIG. 1. The only thing that differentiates the assist device 1A (and body gear 2A and a jacket 20A) shown in FIG. 2 from the assist device 1 (and body gear 2 and a jacket 20) shown in FIG. 1 is the close-fitting belt 25RL. In the following, therefore, the assist device 1 shown in FIG. 1 will be described while the description of the assist device 1A shown in FIG. 2 will be omitted. FIG. 3 is an exploded perspective view of the assist device 1 shown in FIG. 1.

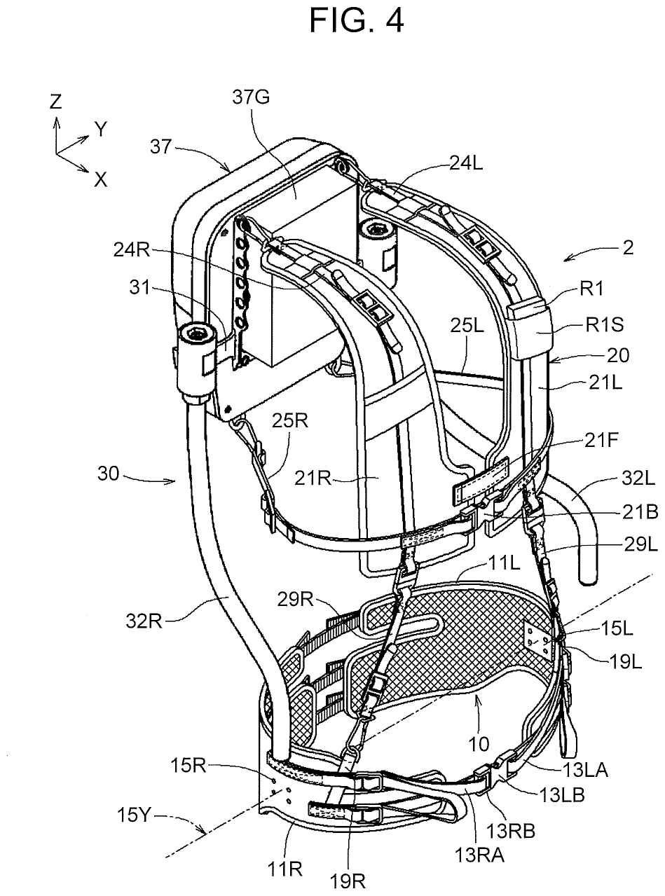

As shown in the exploded perspective view of FIG. 3, the assist device 1 is composed of a hip support 10, the jacket 20, a frame 30, a backpack 37, a cushion 37G, a right actuator unit 4R, a left actuator unit 4L, etc. The hip support 10, the jacket 20, the frame 30, the backpack 37, and the cushion 37G compose the body gear 2 (see FIG. 4), and the right actuator unit 4R and the left actuator unit 4L compose an actuator unit 4. The assist device 1 further has a manipulation unit R1 (so-called remote controller) that is used by the person being assisted to adjust a motion mode (lowering assistance, lifting assistance, etc.), a gain in an assisting torque, and a speed with which the amount of assisting torque is increased, or to check the adjusted state etc., and a housing part R1S that houses the manipulation unit R1.

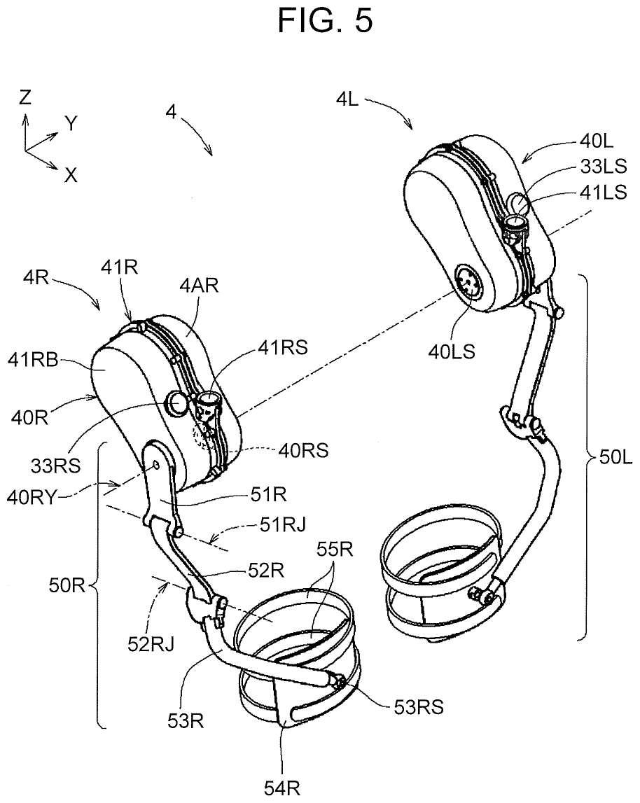

The body gear 2 (see FIG. 4) is worn at least around the hips of the person being assisted. The right actuator unit 4R and the left actuator unit 4L (see FIG. 5) are attached to the body gear 2 and worn on the thighs of the person being assisted to assist the person being assisted in moving his or her thighs relative to the hips or moving his or her hips relative to the thighs. In the following, the body gear 2 and the actuator unit 4 will be described in this order.

As shown in FIG. 3 and FIG. 4, the body gear 2 has: the hip support 10 worn around the hips of the person being assisted; the jacket 20 worn around the shoulders and the chest of the person being assisted; the frame 30 to which the jacket 20 is connected; and the backpack 37 and the cushion 37G both mounted on the frame 30. The frame 30 is disposed on the back and around the hips of the person being assisted.

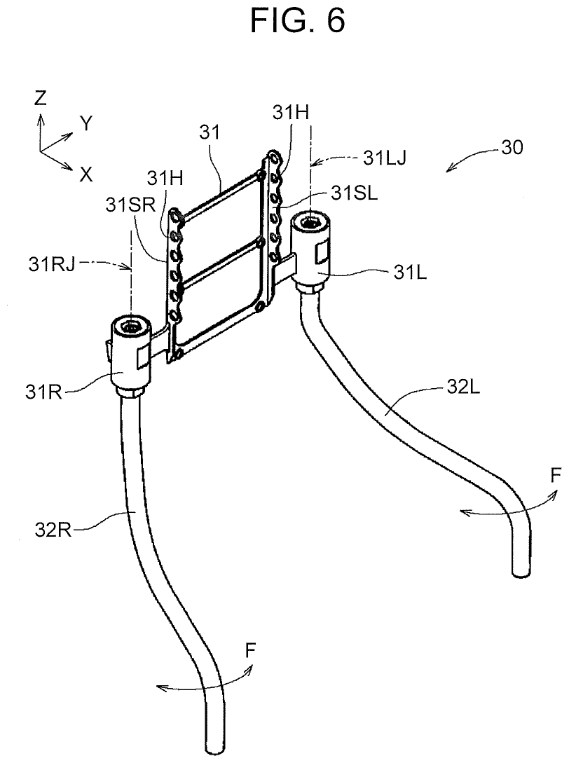

As shown in FIG. 3 and FIG. 6, the frame 30 has a main frame 31, a right sub-frame 32R, a left sub-frame 32L, etc. As shown in FIG. 6, the main frame 31 has support bodies 31SR, 31SL each having a plurality of belt connection holes 31H disposed in an up-down direction, and a connector 31R (right turning shaft) and a connector 31L (left turning shaft). The right sub-frame 32R is connected at one end (upper end) to the connector 31R, and the left sub-frame 32L is connected at one end (upper end) to the connector 31L. The connector 31R is a so-called cylindrical damper and has coaxially disposed inner cylinder and outer cylinder, with a cylindrical elastic body disposed between the inner cylinder and the outer cylinder. The outer cylinder is fixed to the main frame 31, and the right sub-frame 32R is fixed at the one end (upper end) to the inner cylinder. Similarly, an outer cylinder of the connector 31L is fixed to the main frame 31, and the left sub-frame 32L is fixed at the one end (upper end) to an inner cylinder. Thus, the right sub-frame 32R can turn around a turning axis 31RJ and the left sub-frame 32L can turn around a turning axis 31LJ.

As shown in FIG. 1, the right sub-frame 32R is connected (fixed) at a lower end to a connector 41RS of the right actuator unit 4R, and the left sub-frame 32L is connected (fixed) at a lower end to a connector 41LS of the left actuator unit 4L.

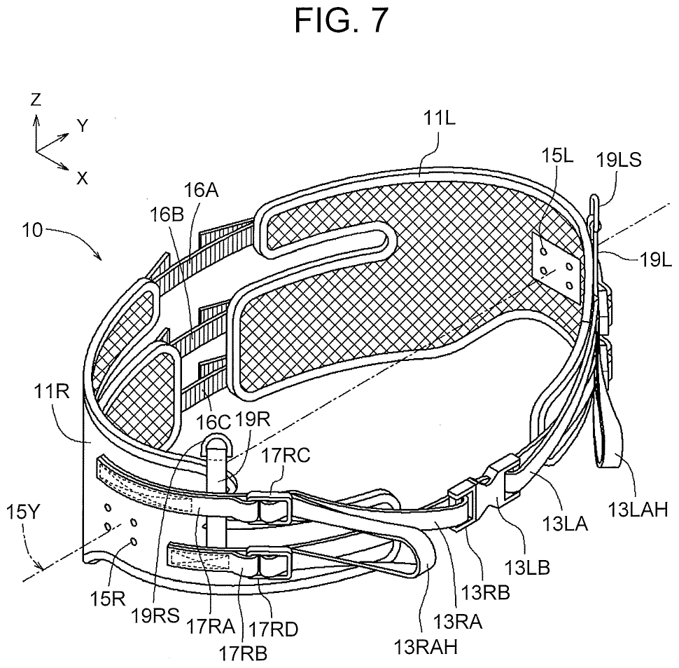

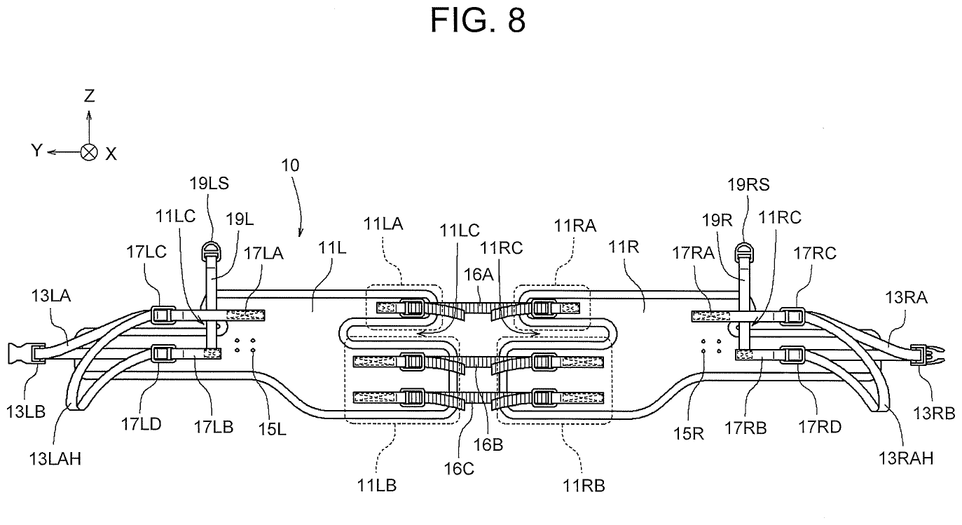

As shown in FIG. 7 and FIG. 8, the hip support 10 has a right hip-worn part 11R worn around the hip on the right side of the person being assisted, and a left hip-worn part 11L worn around the hip on the left side of the person being assisted. As shown in FIG. 8, the right hip-worn part 11R and the left hip-worn part 11L are connected to each other by a back hip belt 16A, an upper buttock belt 16B, and a lower buttock belt 16C.

As shown in FIG. 1 and FIG. 3, the hip support 10 has a coupling belt 19R with a coupling ring 19RS that is coupled to a coupling portion 29RS of the jacket 20, and a coupling belt 19L with a coupling ring 19LS that is coupled to a coupling portion 29LS of the jacket 20. As shown in FIG. 3, the hip support 10 further has mounting holes 15R used to connect the hip support 10 to a coupling portion 40RS of the right actuator unit 4R, and mounting holes 15L used to connect the hip support 10 to a coupling portion 40LS of the left actuator unit 4L, respectively at positions at which the hip support 10 intersects with an imaginary turning axis 15Y.

As shown in FIG. 8, a cutout 11RC is formed in the right hip-worn part 11R, at a position corresponding to the back side of the person being assisted, and the right hip-worn part 11R is thereby divided into a right hip portion 11RA and a right buttock portion 11RB. A cutout 11LC is formed in the left hip-worn part 11L, at a position corresponding to the back side of the person being assisted, and the left hip-worn part 11L is thereby divided into a left hip portion 11LA and a left buttock portion 11LB.

As shown in FIG. 7 and FIG. 8, the hip support 10 has various length-adjustable belts etc. that allow the hip support 10 to closely fit around the hips of the person being assisted without becoming displaced, including a right hip fastening belt 13RA, a hip belt retaining member 13RB (hip buckle), a left hip fastening belt 13LA, a hip belt retaining member 13LB (hip buckle), a right upper pelvis belt 17RA, a right lower pelvis belt 17RB, a left upper pelvis belt 17LA, a left lower pelvis belt 17LB, a right upper belt retaining member 17RC (right upper adjuster), a right lower belt retaining member 17RD (right lower adjuster), a tensioning portion 13RAH, a left upper belt retaining member 17LC (left upper adjuster), a left lower belt retaining member 17LD (left lower adjuster), and a tensioning portion 13LAH.

As shown in FIG. 3, the backpack 37 is mounted on the main frame 31 that forms an upper end part of the frame 30. A right shoulder belt 24R, the right side belt 25R, a left shoulder belt 24L, and the left side belt 25L are connected to the main frame 31 or the backpack 37.

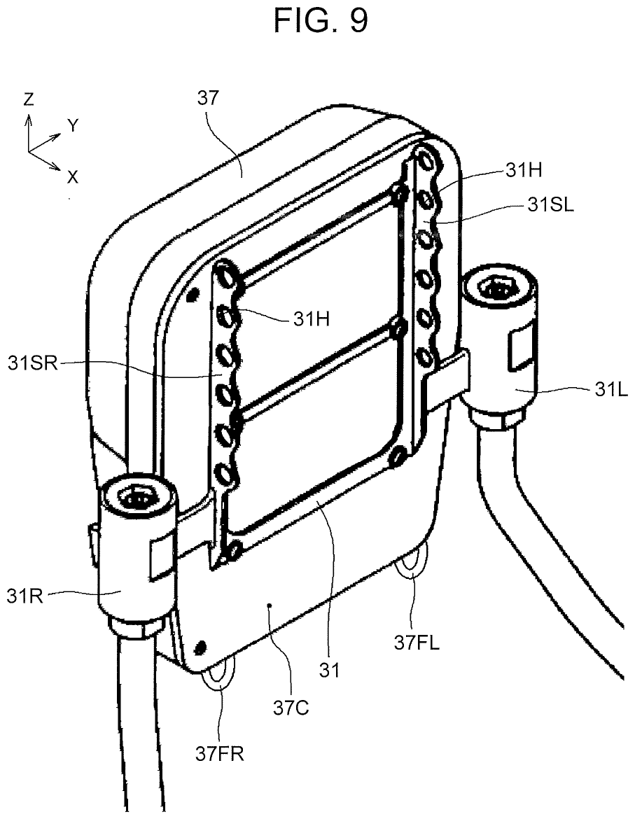

As shown in FIG. 9 and FIG. 10, the backpack 37 has a simple box shape, and houses a controller, a power source unit, communication means, etc. As shown in FIG. 9, the backpack 37 has a back support 37C on the side of the main frame 31. The back support 37C is fixed to the main frame 31. The support bodies 31SR, 31SL each having the belt connection holes 31H (corresponding to belt connectors) disposed in the up-down direction are provided in the main frame 31, at positions facing the back sides of both shoulders of the person being assisted. The belt connection holes 31H (belt connectors) are provided to allow the position in a height direction of the jacket 20 relative to the frame 30 to be adjusted according to the physical size of the person being assisted. Thus, the height of the jacket 20 can be adjusted to an appropriate position according to the physical size of the person being assisted.

Even when the upper body of the person being assisted leans forward, the actuator unit (4R, 4L) that outputs an assisting torque can be appropriately supported if the cushion 37G (or the back support 37C) that comes into contact with the back of the person being assisted is elongated in a direction from the shoulders to the hips of the person being assisted. Moreover, even when the upper body of the person being assisted leans rightward or leftward, the actuator unit (4R, 4L) that outputs an assisting torque can be more appropriately supported (supported with higher rigidity) as the cushion 37G (or the back support 37C) comes into contact with the person being assisted, centered at a bend in his or her back.

As shown in FIG. 10, a belt connector 24RS of the right shoulder belt 24R is connected to one of the belt connection holes 3114 (belt connectors) of the support body 31RS. Similarly, as shown in FIG. 10, a belt connector 24LS of the left shoulder belt 24L is connected to one of the belt connection holes 31H (belt connectors) of the support body 31SL. Alternatively, the support bodies 31SR, 31SL may be provided in the backpack 37.

As shown in FIG. 9 and FIG. 10, belt connectors 37FR, 37FL are respectively provided on right and left sides of a lower end of the backpack 37. As shown in FIG. 10, a belt connector 25RS of the right side belt 25R is connected to the belt connector 37FR. Similarly, as shown in FIG. 10, a belt connector 25LS of the left side belt 25L is connected to the belt connector 37FL. Alternatively, the belt connectors 37FR, 37FL may be provided in the main frame 31.

As shown in FIG. 4, the jacket 20 has a right chest-worn part 21R worn on the right-side chest of the person being assisted, and a left chest-worn part 21L worn on the left-side chest of the person being assisted. The right chest-worn part 21R is connected to the left chest-worn part 21L, for example, by a touch-and-close fastener 21F and a buckle 21B, which allows the person being assisted to easily put on and take off the jacket 20.

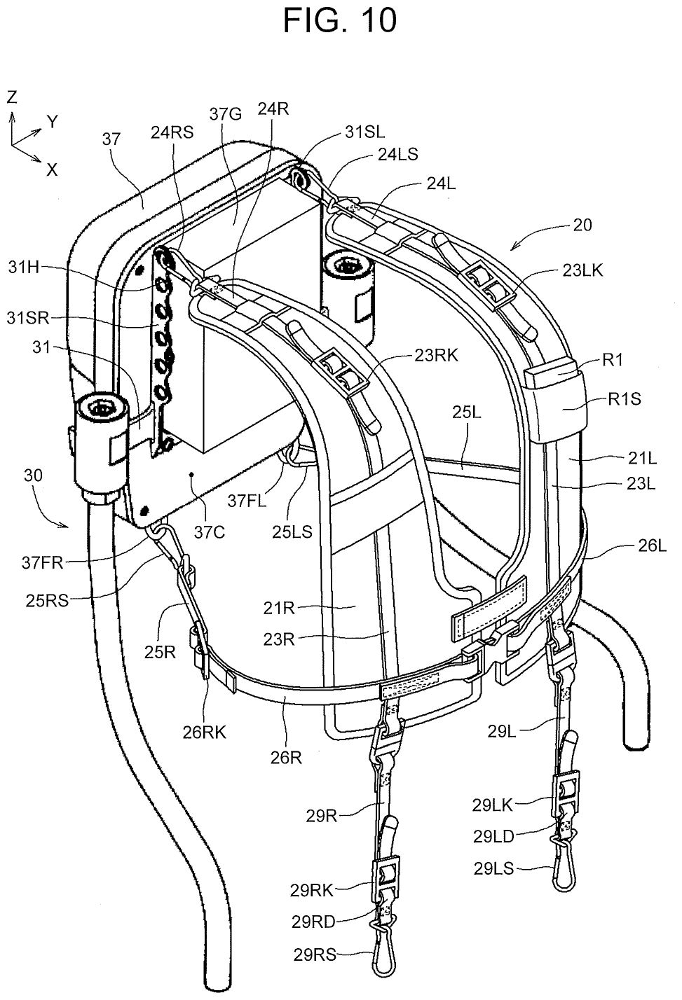

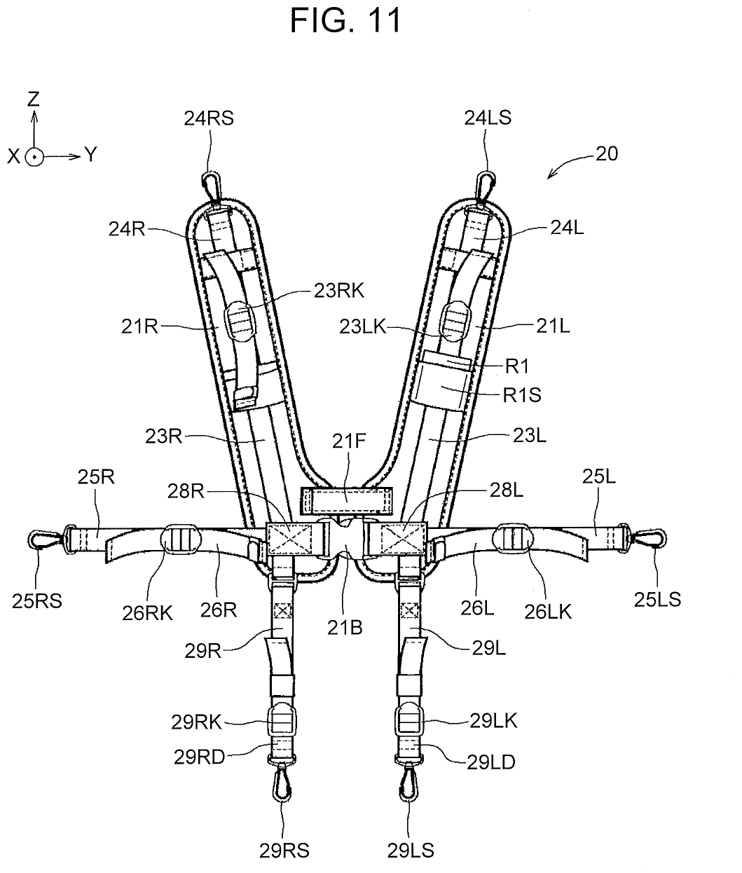

The right chest-worn part 21R has the right shoulder belt 24R and the belt connector 24RS connected to the belt connection hole 31H of the main frame 31 (or the backpack 37), and the right side belt 25R and the belt connector 25RS connected to the belt connector 37FR of the backpack 37 (or the main frame 31). The left chest-worn part 21L has the left shoulder belt 24L and the belt connector 24LS connected to the main frame 31 (or the backpack 37), and the left side belt 25L and the belt connector 25LS connected to the belt connector 37FL of the backpack 37 (or the main frame 31). As shown in FIG. 11, the right chest-worn part 21R has a coupling belt 29R and the coupling portion 29RS by which the right chest-worn part 21R is coupled to the right hip-worn part 11R, and the left chest-worn part 21L has a coupling belt 29L and the coupling portion 29LS by which the left chest-worn part 21L is coupled to the left hip-worn part 11L.

As shown in FIG. 10 and FIG. 11, the jacket 20 has various length-adjustable belts etc. that allow the jacket 20 to closely fit around the chest of the person being assisted without becoming displaced, including a fixing portion 28R, a fixing portion 28L, a right shoulder belt 23R, a right shoulder belt retaining member 23RK (right shoulder adjuster), a left shoulder belt 23L, a left shoulder belt retaining member 23LK (left shoulder adjuster), a right side belt 26R, a right side belt retaining member 26RK (right side adjuster), a left side belt 26L, and a left side belt retaining member 26LK (left side adjuster).

FIG. 5 shows the external appearance of the right actuator unit 4R and the left actuator unit 4L shown in FIG. 3. Since the left actuator unit 4L is symmetrical with the right actuator unit 4R in a lateral direction, the description of the left actuator unit 4L will be omitted from the following description.

As shown in FIG. 5, the right actuator unit 4R has a torque generation part 40R and an output link 50R that is a torque transmission part. The torque generation part 40R has an actuator base 41R, a cover 41RB, and a coupling base 4AR. As shown in FIG. 5, the output link 50R is worn on a body part to be assisted (in this case, the thigh) and turns around a joint (in this case, the hip joint) of the body part to be assisted (in this case, the thigh). An assisting torque that is transmitted through the output link 50R to assist the turning of the body part to be assisted is generated by an electric motor (actuator) inside the torque generation part 40R.

The output link 50R has an assist arm 51R (corresponding to a first link), a second link 52R, a third link 53R, and a thigh-worn part 54R (corresponding to a body holding part). The assist arm 51R is turned around a turning axis 40RY by a combined torque that combines the assisting torque generated by the electric motor inside the torque generation part 40R and a person being assisted-exerted torque resulting from the person being assisted moving his or her thigh. The second link 52R is connected at one end to a leading end of the assist arm 51R so as to be able to turn around a turning axis 51RJ, and the third link 53R is connected at one end to the other end of the second link 52R so as to be able to turn around a turning axis 52RJ. The thigh-worn part 54R is connected to the other end of the third link 53R through a third joint 53RS (in this case, a spherical joint).

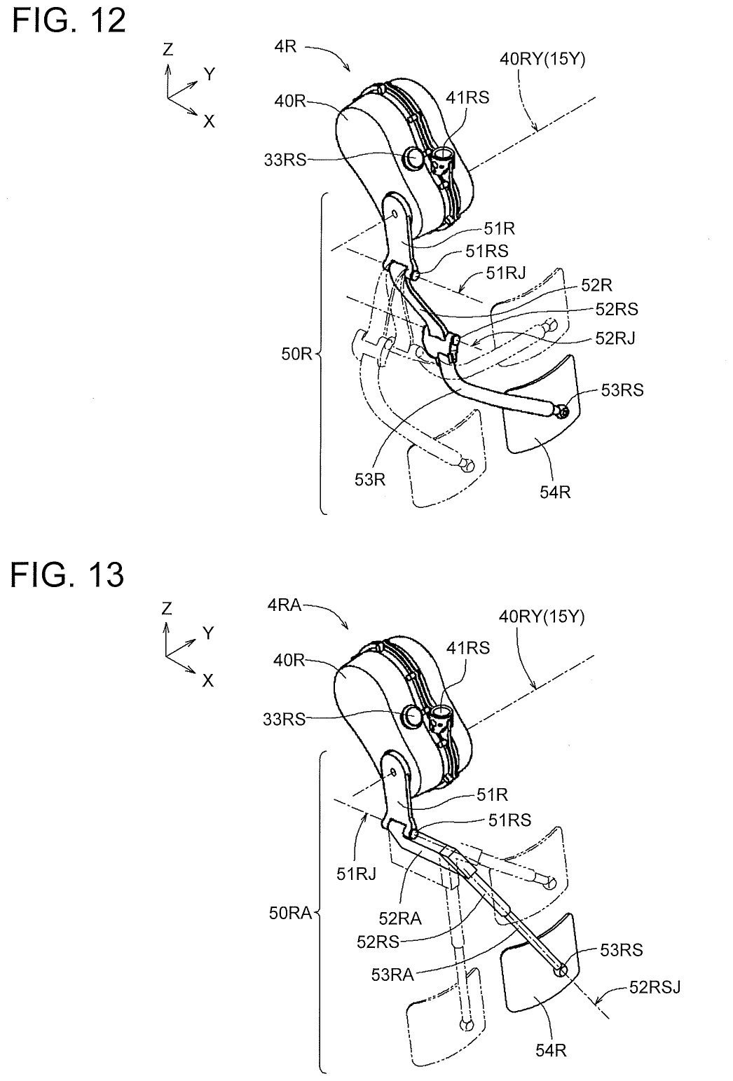

Next, the link mechanism of the right actuator unit 4R will be described in detail by using FIG. 5 and FIG. 12 to FIG. 18. As examples of the link mechanism, the example of an output link 50R shown in FIG. 12 and the example of an output link 50RA shown in FIG. 13 will be described.

The output link 50R shown in FIG. 12 is composed of a plurality of coupling members, namely, the assist arm 51R (corresponding to the first link), the second link 52R, the third link 53R, and the thigh-worn part 54R (corresponding to the body holding part) that are coupled to one another by joints. Thigh belts 55R shown in FIG. 14 are omitted from the thigh-worn part 54R shown in FIG. 12.

The second link 52R is coupled at the one end to the leading end of the assist arm 51R by a first joint 51RS so as to be able to turn around the turning axis 51RJ. The first joint 51RS has a coupling structure with one degree of freedom that allows the second link 52R to turn around the turning axis 51RJ relative to the assist arm 51R.

The third link 53R is coupled at the one end to the other end of the second link 52R by a second joint 52RS so as to be able to turn around the turning axis 52RJ. The second joint 52RS has a coupling structure with one degree of freedom that allows the third link 53R to turn around the turning axis 52RJ relative to the second link 52R.

The third link 53R is coupled at the other end to the thigh-worn part 54R by the third joint 53RS (a spherical joint in the example of FIG. 14). Accordingly, the third joint 53RS between the third link and the thigh-worn part 54R (body holding part) has a coupling structure with three degrees of freedom. Thus, the total number of degrees of freedom of the output link 50R shown in FIG. 12 is: 1+1+3=5.

However, the total number of degrees of freedom of the output link 50R may be any number not smaller than three. For example, as shown in FIG. 14, the third joint 53RS may be configured so as to allow the thigh-worn part 54R to turn around a turning axis 53RJ relative to the other end of the third link 53R. In the example of FIG. 14, the third joint 53RS has a coupling structure with one degree of freedom that allows the thigh-worn part 54R to turn around the turning axis 53RJ relative to the third link 53R. Thus, with the first joint 51RS and the second joint 52RS each having one degree of freedom, the total number of degrees of freedom of the output link in this case is: 1+1+1=3. It is preferable that a stopper that limits the range of turning of the second link or the third link be provided.

As shown in FIG. 14, the body holding part is composed of the thigh-worn part 54R that is coupled to the third link 53R and worn on the thigh of the person being assisted, and the stretchable thigh belts 55R that are provided on the thigh-worn part 54R so as to encircle the thigh of the person being assisted. The thigh belt 55R is formed by a stretchy elastic body, and one end side of the thigh belt 55R is fixed to the thigh-worn part 54R while the other end side forms a touch-and-close fastener 55RM. A touch-and-close fastener 54RM is provided in the thigh-worn part 54R, at a position facing the other end side of the thigh belt 55R.

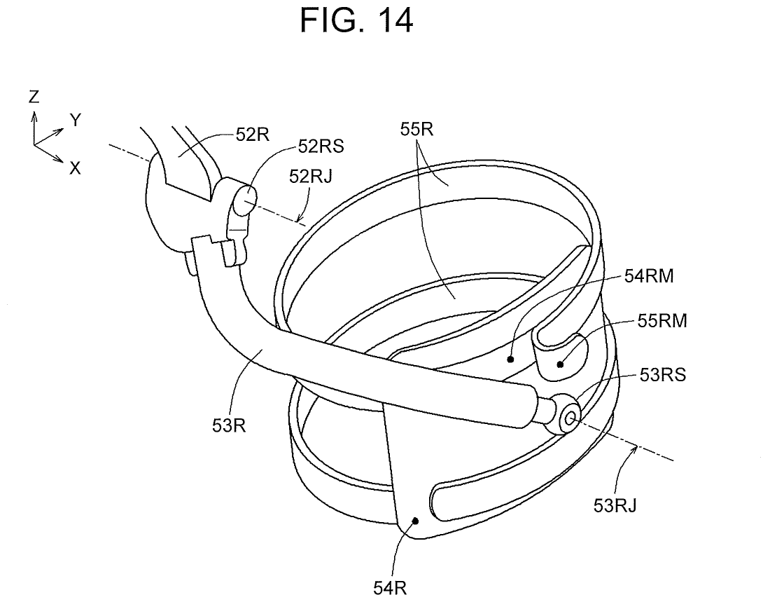

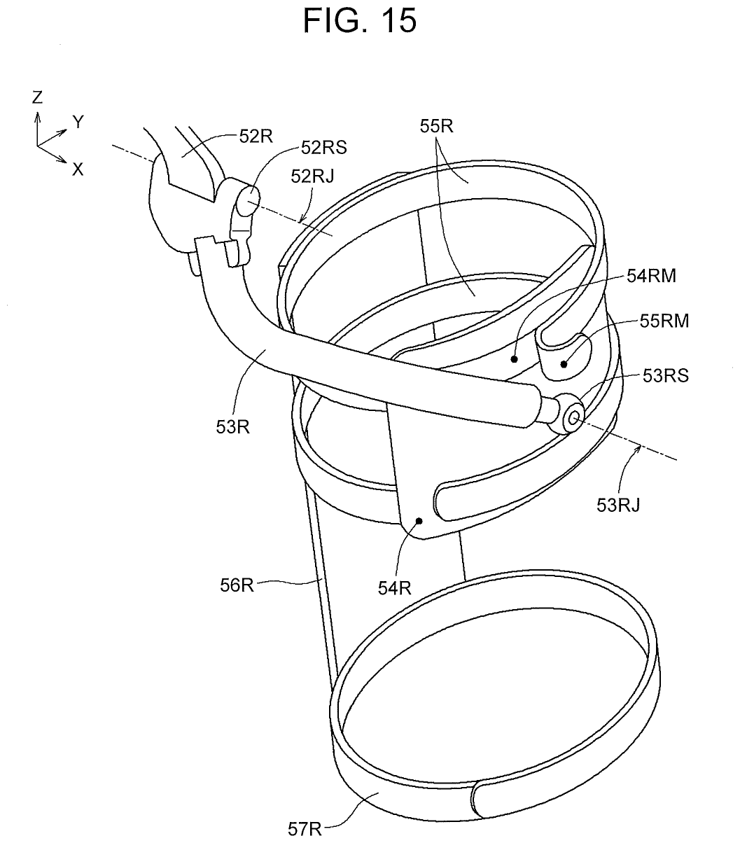

While FIG. 14 shows an example in which the body holding part is composed of the thigh-worn part 54R and the thigh belts 55R, FIG. 15 shows an example in which the body holding part is composed of the thigh-worn part 54R, the thigh belts 55R, and an under-knee belt 57R. As shown in FIG. 15, the thigh belts 55R are provided on the thigh-worn part 54R so as to encircle the thigh above the knee of the person being assisted. The under-knee belt 57R is provided so as to encircle a part under the knee of the person being assisted. The under-knee belt 57R is made of the same material as the thigh belts 55R, has a touch-and-close fastener as with the thigh belts 55R, and is closely fitted around the part under the knee. On the back side of the knee of the person being assisted, the thigh belts 55R and the under-knee belt 57R are coupled together by a coupling member 56R extending in a direction from the thigh toward the foot of the person being assisted. The coupling member 56R is disposed behind the knee of the person being assisted, and is made of a material that allows the coupling member 56R to bend as the person being assisted bends and unbends his or her knee. Thus, the thigh belts 55R are retained in close contact with the part above the knee of the person being assisted, while the under-knee belt 57R is retained in close contact with the part under the knee of the person being assisted.

The output link 50RA shown in FIG. 13 is composed of a plurality of coupling members, namely, the assist arm 51R (corresponding to the first link), a second link 52RA (and the second joint 52RS), a third link 53RA, and the thigh-worn part 54R (corresponding to the body holding part) that are coupled to one another by joints. The thigh belts 55R shown in FIG. 14 are omitted from the thigh-worn part 54R shown in FIG. 13.

The second link 52RA is coupled at an end to the leading end of the assist arm 51R by the first joint 51RS so as to be able to turn around the turning axis 51RJ. The first joint 51RS has a coupling structure with one degree of freedom that allows the second link 52RA to turn around the turning axis 51RJ relative to the assist arm 51R.

The second link 52RA and the second joint 52RS are integrated with each other, and the third link 53RA capable of sliding back and forth along a sliding axis 52RSJ that is an axis in a longitudinal direction is coupled at one end to the second link 52RA by the second joint 52RS. The second joint 52RS has a coupling structure with one degree of freedom that allows the third link 53RA to slide along the sliding axis 52RSJ relative to the second link 52RA.

The third link 53RA is coupled to the thigh-worn part 54R by the third joint 53RS (a spherical joint in the example of FIG. 13). Accordingly, the third joint 53RS between the third link 53RA and the thigh-worn part 54R (body holding part) has a coupling structure with three degrees of freedom. Thus, the total number of degrees of freedom of the output link 50RA shown in FIG. 13 is: 1+1+3=5.

Since the total number of degrees of freedom may be any number not smaller than three, as shown in FIG. 14, the third joint may have a coupling structure with one degree of freedom that allows the thigh-worn part 54R to turn around the turning axis 53RJ. It is preferable that a stopper that limits the range of turning of the second link 52RA or the range of sliding of the third link 53RA be provided.

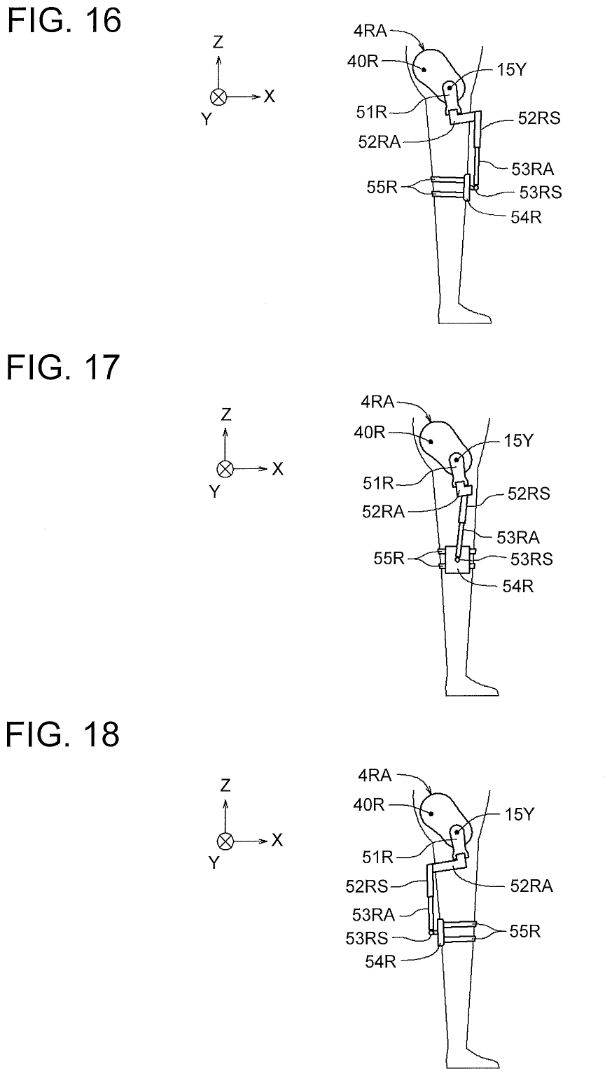

FIG. 16 to FIG. 18 are views illustrating examples in which, in the link mechanism shown in FIG. 12, the third joint 53RS that is a coupling portion between the third link 53RA and the thigh-worn part 54R is disposed on a front surface of the thigh of the person being assisted (FIG. 16), on an outer lateral surface of the thigh of the person being assisted (FIG. 17), and on a back surface of the thigh of the person being assisted (FIG. 18). The position of the third joint 53RS may be any one of the front surface, the lateral surface, and the back surface of the thigh of the person being assisted.

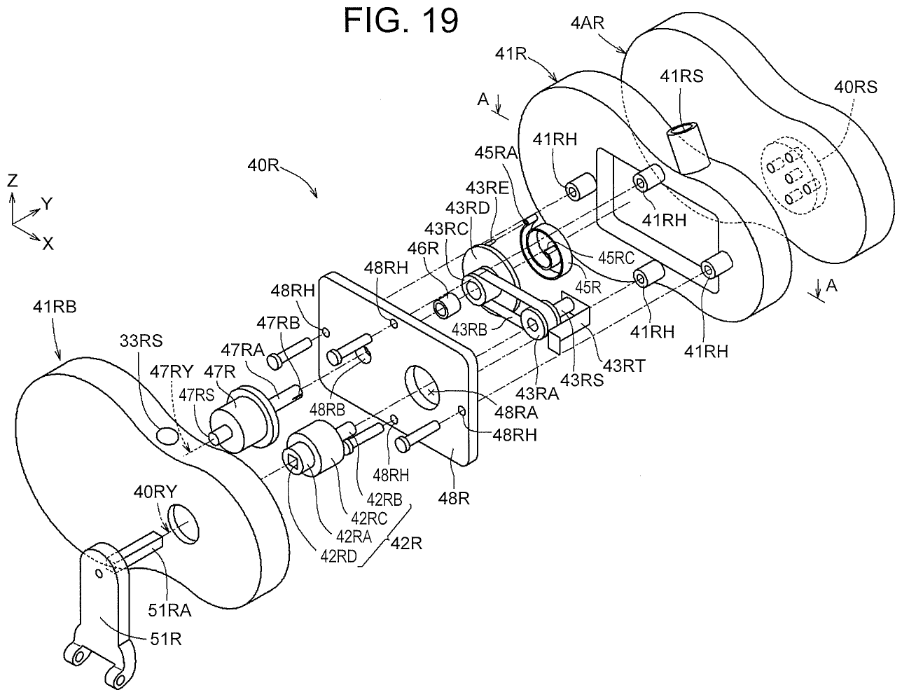

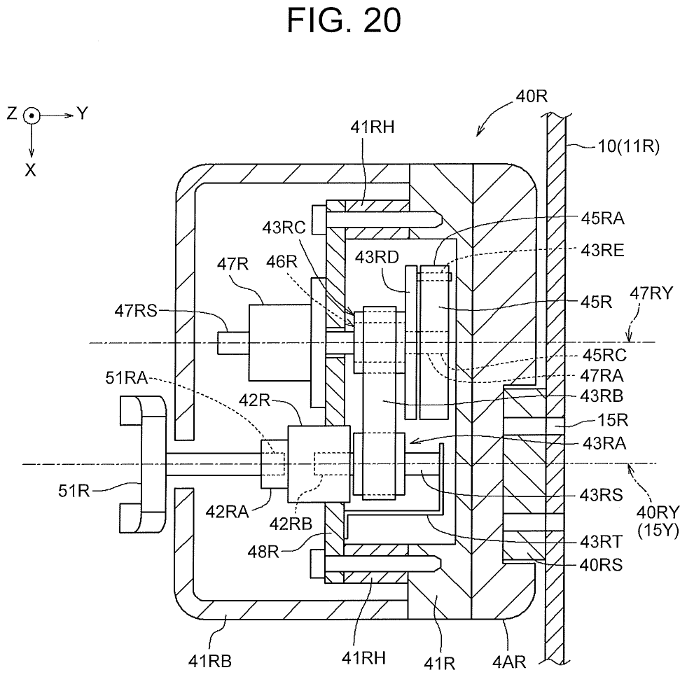

Next, members housed inside the cover 41RB of the torque generation part 40R (see FIG. 5) will be described by using FIG. 19 and FIG. 20. FIG. 20 is a sectional view taken along line A-A in FIG. 19. As shown in FIG. 19 and FIG. 20, the cover 41RB houses a speed reducer 42R, a pulley 43RA, a transmission belt 43RB, a pulley 43RC having a flange 43RD, a spiral spring 45R, a bearing 46R, an electric motor 47R (actuator), a sub-frame 48R, etc. The assist arm 51R having a shaft 51RA is disposed on an outer side of the cover 41RB.

Outlet ports 33RS, 33LS (connection ports) for an actuator driving cable, a control cable, and a communication cable are provided in the actuator units (4R, 4L) at portions near the frame 30. The cables (not shown) connected to the cable outlet ports 33RS, 33LS are disposed along the frame 30 and connected to the backpack 37.

As shown in FIG. 20, the torque generation part 40R has the actuator base 41R on which the sub-frame 48R having the electric motor 47R etc. installed thereon is mounted, the cover 41RB mounted on one side of the actuator base 41R, and the coupling base 4AR mounted on the other side of the actuator base 41R. The coupling portion 40RS capable of turning around the turning axis 40RY is provided on the coupling base 4AR.

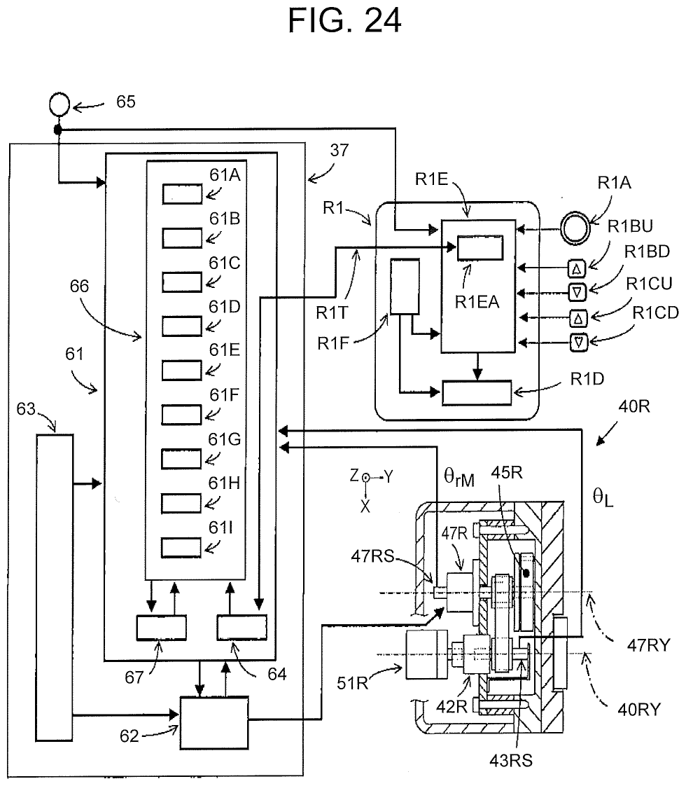

As shown in FIG. 19 and FIG. 20, output link turning angle detection means 43RS (turning angle sensor etc.) that detects a turning angle of the assist arm 51R relative to the actuator base 41R is connected to the pulley 43RA that is connected to a speed increasing shaft 42RB of the speed reducer 42R. The output link turning angle detection means 43RS is, for example, an encoder or an angle sensor, and outputs a detection signal according to the rotation angle to a controller 61 (see FIG. 24). The electric motor 47R is provided with motor rotation angle detection means 47RS capable of detecting a rotation angle of a motor shaft (corresponding to an output shaft). The motor rotation angle detection means 47RS is, for example, an encoder or an angle sensor, and outputs a detection signal according to the rotation angle to the controller 61 (see FIG. 24).

As shown in FIG. 19, the sub-frame 48R has a through-hole 48RA in which a speed reducer housing 42RC of the speed reducer 42R is fixed, and a through-hole 48RB through which an output shaft 47RA of the electric motor 47R is passed. The shaft 51RA of the assist arm 51R is fitted in a hole 42RD of a speed reducing shaft 42RA of the speed reducer 42R, and the speed reducer housing 42RC of the speed reducer 42R is fixed to the through-hole 48RA of the sub-frame 48R. Thus, the assist arm 51R is supported so as to be able to turn around the turning axis 40RY relative to the actuator base 41R, and turns integrally with the speed reducing shaft 42RA. The electric motor 47R is fixed to the sub-frame 48R, and the output shaft 47RA is passed through the through-hole 48RB of the sub-frame 48R. The sub-frame 48R is fixed to mounting portions 41RH of the actuator base 41R with fastening members, such as bolts.

As shown in FIG. 19, the pulley 43RA is connected to the speed increasing shaft 42RB of the speed reducer 42R, and the output link turning angle detection means 43RS is connected to the pulley 43RA. A support member 43RT fixed to the sub-frame 48R is connected to the output link turning angle detection means 43RS. Thus, the output link turning angle detection means 43RS can detect the turning angle of the speed increasing shaft 42RB relative to the sub-frame 48R (i.e., relative to the actuator base 41R). The turning angle of the assist arm 51R is a turning angle having been increased by the speed increasing shaft 42RB of the speed reducer 42R, and therefore the output link turning angle detection means 43RS and the controller can detect the turning angle of the assist atm 51R with higher resolution. By detecting the turning angle of the output link with higher resolution, the controller can execute control with higher accuracy. The shaft 51RA of the assist arm 51R, the speed reducer 42R, the pulley 43RA, and the output link turning angle detection means 43RS are disposed coaxially along the turning axis 40RY.

The speed reducer 42R has a set speed reduction ratio n (1<n), and turns the speed increasing shaft 42RB a turning angle nθ when the speed reducing shaft 42RA is turned a turning angle θ. When the speed increasing shaft 42RB is turned the turning angle nθ, the speed reducer 42R turns the speed reducing shaft 42RA the turning angle θ. The transmission belt 43RB is wrapped around the pulley 43RA to which the speed increasing shaft 42RB of the speed reducer 42R is connected and around the pulley 43RC. Accordingly, the person being assisted-exerted torque from the assist arm 51R is transmitted to the pulley 43RC through the speed increasing shaft 42RB, and the assisting torque from the electric motor 47R is transmitted to the speed increasing shaft 42RB through the spiral spring 45R and the pulley 43RC.

The spiral spring 45R has a spring constant Ks, and has a shape of a spiral with an inner end 45RC on a center side and an outer end 45RA on an outer circumferential side. The inner end 45RC of the spiral spring 45R is fitted in a groove 47RB formed in the output shaft 47RA of the electric motor 47R. The outer end 45RA of the spiral spring 45R is wound into a cylindrical shape. A transmission shaft 43RE provided on the flange 43RD of the pulley 43RC is fitted in the outer end 45RA, and the outer end 45RA is supported by the transmission shaft 43RE. (The pulley 43RC is integrated with the flange 43RD and the transmission shaft 43RE). The pulley 43RC is supported so as to be able to turn around a turning axis 47RY, and the transmission shaft 43RE protruding toward the spiral spring 45R is provided near an outer circumferential edge of the flange 43RD integrated with the pulley 43RC. The transmission shaft 43RE is fitted in the outer end 45RA of the spiral spring 45R, and moves the position of the outer end 45RA around the turning axis 47RY. A bearing 46R is provided between the output shaft 47RA of the electric motor 47R and the pulley 43RC. Thus, the output shaft 47RA is not fixed to the pulley 43RC, and the output shaft 47RA can rotate independently of the pulley 43RC. The pulley 43RC is driven to rotate by the electric motor 47R through the spiral spring 45R. In the configuration having been described above, the output shaft 47RA of the electric motor 47R, the bearing 46R, the pulley 43RC having the flange 43RD, and the spiral spring 45R are disposed coaxially along the turning axis 47RY.

The spiral spring 45R accumulates the assisting torque that is transmitted from the electric motor 47R and the person being assisted-exerted torque that results from the person being assisted moving his or her thigh and is transmitted via the assist arm 51R, the speed reducer 42R, the pulley 43RA, and the pulley 43RC, and thus accumulates the combined torque combining the assisting torque and the person being assisted-exerted torque. The combined torque accumulated in the spiral spring 45R turns the assist arm 51R through the pulley 43RC, the pulley 43RA, and the speed reducer 42R. In the configuration having been described above, the output shaft 47RA of the electric motor 47R is connected to the output link (in the case of FIG. 19, the assist arm 51R) through the speed reducer 42R that reduces the rotation angle of the output shaft 47RA.

The combined torque accumulated in the spiral spring 45R is obtained based on an amount of change in angle from a no-load state and the spring constant, for example, based on the turning angle of the assist arm 51R (obtained by the output link turning angle detection means 43RS), the rotation angle of the output shaft 47RA of the electric motor 47R (obtained by the motor rotation angle detection means 47RS), and the spring constant Ks of the spiral spring 45R. The person being assisted-exerted torque is extracted from the obtained combined torque, and an assisting torque according to this person being assisted-exerted torque is output from the electric motor.

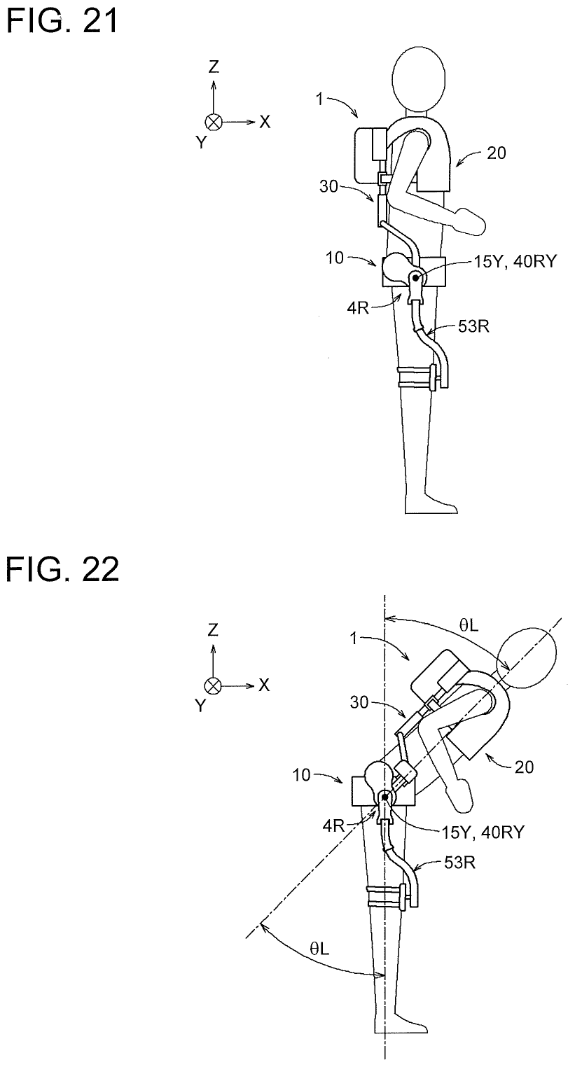

As shown in FIG. 20, the torque generation part 40R of the right actuator unit has the coupling portion 40RS capable of turning around the turning axis 40RY (i.e., the imaginary turning axis 15Y). As shown in FIG. 3 and FIG. 1, the coupling portion 40RS is coupled (fixed) through the mounting holes 15R of the hip support 10 with coupling members, such as bolts. As shown in FIG. 3 and FIG. 1, the right sub-frame 32R of the frame 30 is connected (fixed) at the lower end to the connector 41RS of the right actuator unit 4R. Similarly, the coupling portion 40LS of a torque generation part 40L of the left actuator unit is coupled (fixed) through the mounting holes 15L of the hip support 10 with coupling members, such as bolts, and the left sub-frame 32L of the frame 30 is connected (fixed) at the lower end to the connector 41LS of the left actuator unit 4L. Thus, in FIG. 3, the hip support 10 and the frame 30 are fixed to the torque generation part 40R of the right actuator unit 4R, and the hip support 10 and the frame 30 are fixed to the torque generation part 40L of the left actuator unit 4L. The right actuator unit 4R, the left actuator unit 4L, and the frame 30 are integrated with one another, and are capable of turning relative to the hip support 10 by the coupling portions 40RS, 40LS capable of turning around the imaginary turning axis 15Y (see FIG. 21 and FIG. 22).

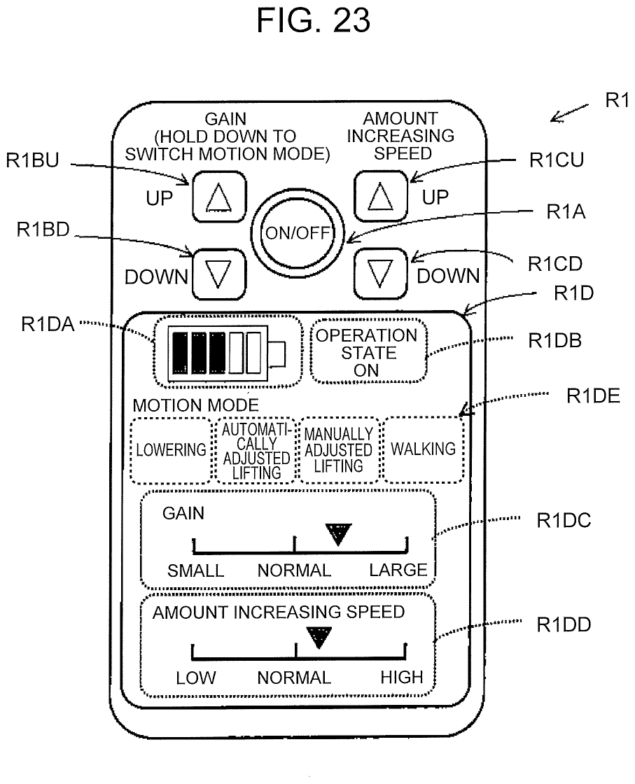

Next, the manipulation unit R1 that allows the person being assisted to easily make adjustments etc. to the assisting state of the assist device 1 will be described by using FIG. 23 to FIG. 25. As shown in FIG. 24, the manipulation unit R1 is connected to the controller 61 inside the backpack 37 (see FIG. 1) through a wired or wireless communication line R1T. A controller R1E of the manipulation unit R1 is capable of transmitting and receiving information to and from the controller 61 through communication means R1EA, and the controller 61 is capable of transmitting and receiving information to and from the controller R1E inside the manipulation unit R1 through communication means 64. As shown in FIG. 1, when not manipulating the manipulation unit R1, the person being assisted can house the manipulation unit R1, for example, in the housing part R1S that is a pocket or the like provided in the jacket 20 (see FIG. 1).

As shown in FIG. 23, the manipulation unit R1 has a main manipulation part R1A, a gain upward manipulation part R1BU, a gain downward manipulation part R1BD, an amount increasing speed upward manipulation part R1CU, an amount increasing speed downward manipulation part R1CD, a display part R1D, etc. The gain upward manipulation part R1BU and the gain downward manipulation part R1BD are an example of the “gain changing part,” and the amount increasing speed upward manipulation part R1CU and the amount increasing speed downward manipulation part R1CD are an example of the “amount increasing speed changing part.” As shown in FIG. 24, the controller R1E, a manipulation unit power source R1F, etc. are provided inside the manipulation unit R1. To prevent an accidental manipulation of the manipulation unit R1 while the manipulation unit R1 is housed inside the housing part R1S (see FIG. 1), it is preferable that the main manipulation part R1A, the gain upward manipulation part R1BU, the gain downward manipulation part R1BD, the amount increasing speed upward manipulation part R1CU, and the amount increasing speed downward manipulation part R1CD do not protrude from a plane in which these parts are disposed.

The main manipulation part R1A is a switch that is manipulated by the person being assisted to start and stop assisting control executed by the assist device 1. As shown in FIG. 24, a main power switch 65 used to start and stop the (entire) assist device 1 itself is provided, for example, in the backpack 37. When the main power switch 65 is manipulated to the ON position, the controller 61 and the controller R1E are started, and when the main power switch 65 is manipulated to the OFF position, the operation of the controller 61 and the controller R1E is stopped. As shown in FIG. 23, whether the current operation state of the assist device is ON (in operation) or OFF (shut-down) is displayed, for example, in a display area R1DB of the display part R1D of the manipulation unit R1.

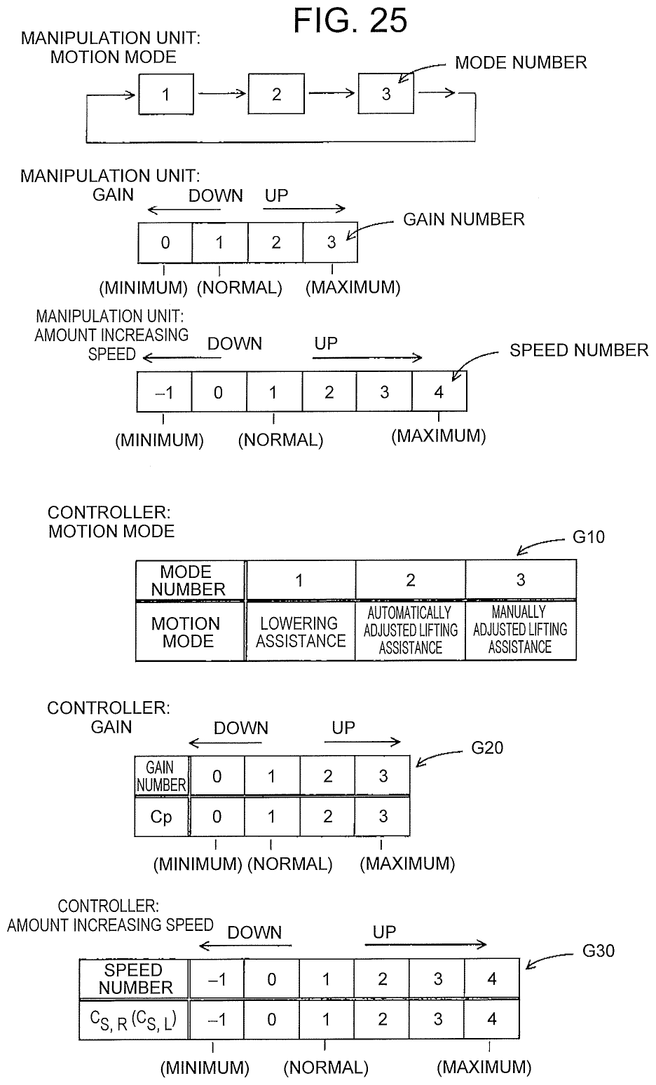

The gain upward manipulation part R1BU is a switch that is manipulated by the person being assisted to increase the gain in the assisting torque generated by the assist device, and the gain downward manipulation part R1BD is a switch that is manipulated by the person being assisted to reduce the gain in the assisting torque generated by the assist device. For example, as shown in “Manipulation Unit: Gain” in FIG. 25, the controller R1E increases a stored gain number by one each time the gain upward manipulation part R1BU is manipulated, and decreases the gain number by one each time the gain downward manipulation part R1BD is manipulated. While FIG. 25 shows an example in which the gain number has four numbers from 0 to 3, the gain number is not limited to four numbers. As shown in FIG. 23, the controller R1E displays a content according to the current gain number, for example, in a display area R1DC of the display part RID of the manipulation unit R1.

When the gain upward manipulation part R1BU is held down, for example, for 5 [sec] or longer, the gain upward manipulation part R1BU functions as a motion mode switch. When the gain upward manipulation part R1BU is held down, the motion mode (mode number) switches sequentially from 1 (lowering assistance) to 2 (automatically adjusted lifting assistance) to 3 (manually adjusted lifting assistance), as shown in “Manipulation Unit: Motion Mode” in FIG. 25, each time the gain upward manipulation part R1BU is pressed. In this case, the gain upward manipulation part R1BU is an example of the “motion switching part.” As shown in FIG. 23, the controller R1E displays a content according to the current motion mode, for example, in a display area RIDE of the display part R1D of the manipulation unit R1. “Walking” mode is a motion mode which cannot be specified through the gain upward manipulation part R1BU but to which the motion mode is automatically switched when the controller 61 recognizes that the person being assisted is “walking.”

The amount increasing speed upward manipulation part R1CU is a switch that is manipulated by the person being assisted to increase the speed with which the amount of assisting torque generated by the assist device is increased, and the amount increasing speed downward manipulation part R1CD is a switch that is manipulated by the person being assisted to reduce the speed with which the amount of assisting torque generated by the assist device is increased. For example, as shown in “Manipulation Unit: Amount Increasing Speed” in FIG. 25, the controller R1E increases a stored speed number by one each time the amount increasing speed upward manipulation part R1CU is manipulated, and decreases the speed number by one each time the amount increasing speed downward manipulation part R1CD is manipulated. FIG. 25 shows an example in which the speed number has six numbers from −1 to 4, but the speed number is not limited to six numbers. As shown in FIG. 23, the controller R1E displays a content according to the current speed number, for example, in a display area R1DD of the display part RID of the manipulation unit R1.

The controller R1E of the manipulation unit R1 transmits manipulation information through the communication means R1EA (see FIG. 24) at predetermined time intervals (e.g., several-millisecond to several-hundred-millisecond intervals) or each time one of the main manipulation part R1A, the gain upward manipulation part R1BU, the gain downward manipulation part R1BD, the amount increasing speed upward manipulation part R1CU, and the amount increasing speed downward manipulation part R1CD is manipulated. This manipulation information includes a stop or start command, the mode number, the gain number, the speed number, etc.

Upon receiving the manipulation information, the controller 61 of the backpack 37 stores the received manipulation information, and transmits, through the communication means 64 (see FIG. 24), response information including battery information showing a battery state of the power source unit 63 used to drive the assist device, assistance information showing an assisting state, etc. The battery information included in the response information includes the remaining battery power of the power source unit 63 etc., and the assistance information included in the response information includes, for example, error information showing contents of an abnormality if any abnormality with the assist device has been found. As shown in FIG. 23, the controller R1E displays the remaining battery power, for example, in a display area R1DA of the display part RID of the manipulation unit R1, and if error information is included, displays the error information somewhere in the display part R1D.

Upon receiving the manipulation information from the controller R1E, the controller 61 (see FIG. 24) starts the assist device when the start command is included in the received manipulation information, and stops the assist device when the stop command is included in the received manipulation information. As shown in “Controller: Motion Mode” in FIG. 25, for example, the controller 61 stores the motion mode according to the received mode number. Further, the controller 61 stores the value (0 to 3) of a gain Cₚ according to the gain number, for example, as shown in “Controller: Gain” in FIG. 25, and stores a (right) amount increasing speed Cₛ, _{R} (right speed number: −1 to 4) and a (left) amount increasing speed Cₛ, _{L} (left speed number: −1 to 4) according to the speed number. The motion mode, the gain Cₚ, and the amount increasing speeds Cₛ, _{R}, Cₛ, _{L} are used in a processing procedure to be described later.

As has been described above, the person being assisted can easily make adjustments for obtaining a desired assisting state by manipulating the manipulation unit R1. Moreover, the person being assisted can easily learn the state of the assist device from the remaining battery power, the error information, etc. displayed in the display part RID of the manipulation unit R1. The forms of the various pieces of information displayed in the display part RID are not limited to those in the example of FIG. 23.

As shown in FIG. 24, the controller 61 is housed inside the backpack 37. In the example shown in FIG. 24, the controller 61, a motor driver 62, the power source unit 63, etc. are housed inside the backpack 37. For example, the controller 61 has control means 66 (CPU) and a storage unit 67 (that stores a control program etc.). The controller 61 has an adjustment determination unit 61A, an input processing unit 61B, a torque change amount etc. calculation unit 61C, a motion type determination unit 61D, a selection unit 61E, a lowering assisting torque calculation unit 61F, a lifting assisting torque calculation unit 61G, a walking assisting torque calculation unit 61H, a control command value calculation unit 61I, the communication means 64, etc. to be described later. The motor driver 62 is an electronic circuit that outputs a driving current for driving the electric motor 47R based on a control signal from the controller 61. The power source unit 63 is a lithium battery, for example, and supplies electricity to the controller 61 and the motor driver 62. The operation of the communication means 64 etc. will be described later.

The manipulation information from the manipulation unit R1, a detection signal from the motor rotation angle detection means 47RS (a detection signal according to an actual motor shaft angle θ_{rM} of the electric motor 47R), a detection signal from the output link turning angle detection means 43RS (a detection signal according to an actual link angle θ_{L} of the assist arm 51R), etc. are input into the controller 61. The controller 61 obtains a rotation angle of the electric motor 47R based on the input signals, and outputs a control signal according to the obtained rotation angle to the motor driver 62.

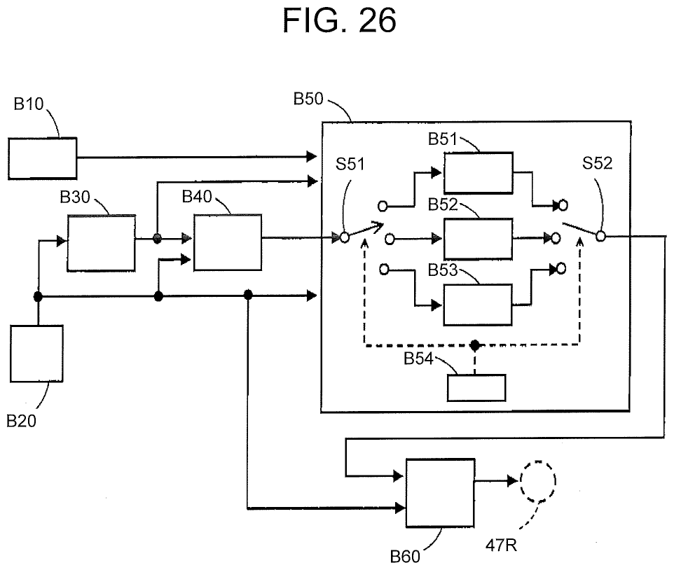

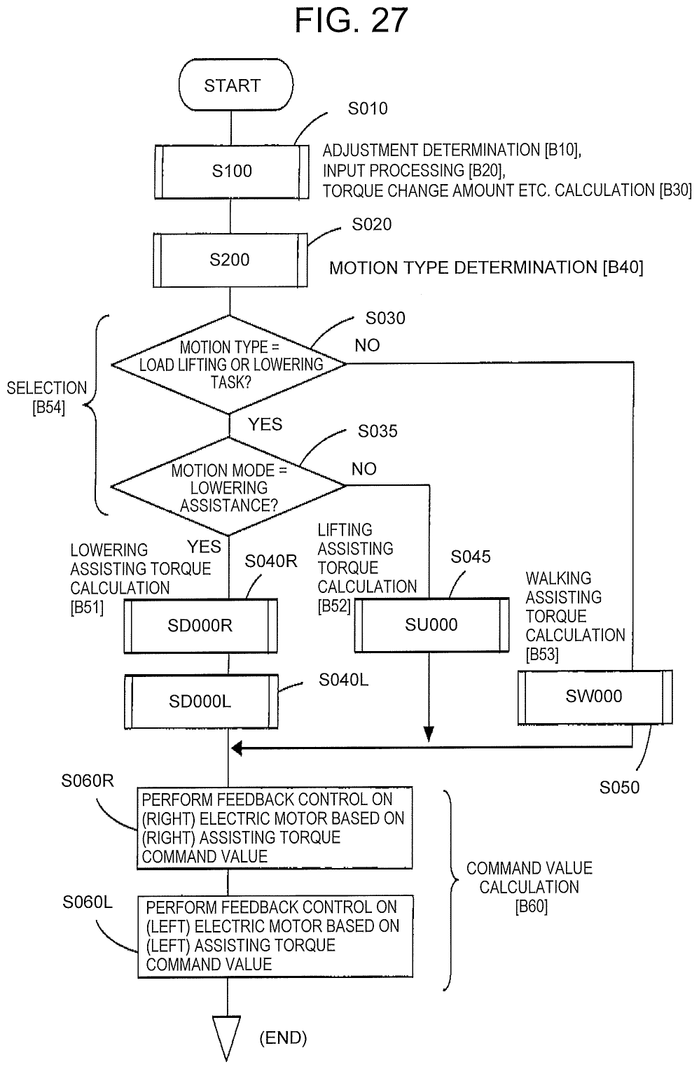

Next, the procedure of a process executed by the controller 61 will be described by using the flowchart shown in FIG. 27 and the control block shown in FIG. 26. The control block shown in FIG. 26 has an adjustment determination block B10, an input processing block B20, a torque change amount etc. calculation block B30, a motion type determination block B40, a selection block B54, a lowering assisting torque calculation block B51, a lifting assisting torque calculation block B52, a walking assisting torque calculation block B53, a control command value calculation block B60, switches S51, S52, etc. A control block B50 shown in FIG. 26 is a control block for determining and selecting lift-up assist mode, bring-down assist more or walking assist mode. Contents of a process executed in each block will be described in accordance with the flowchart shown in FIG. 27.

The flowchart shown in FIG. 27 shows the procedure of the process of controlling the (right) actuator unit 4R and the (left) actuator unit 4L. The process shown in FIG. 27 is started at predetermined time intervals (e.g., several-millisecond intervals), and when this process is started, the controller 61 moves to step S010.

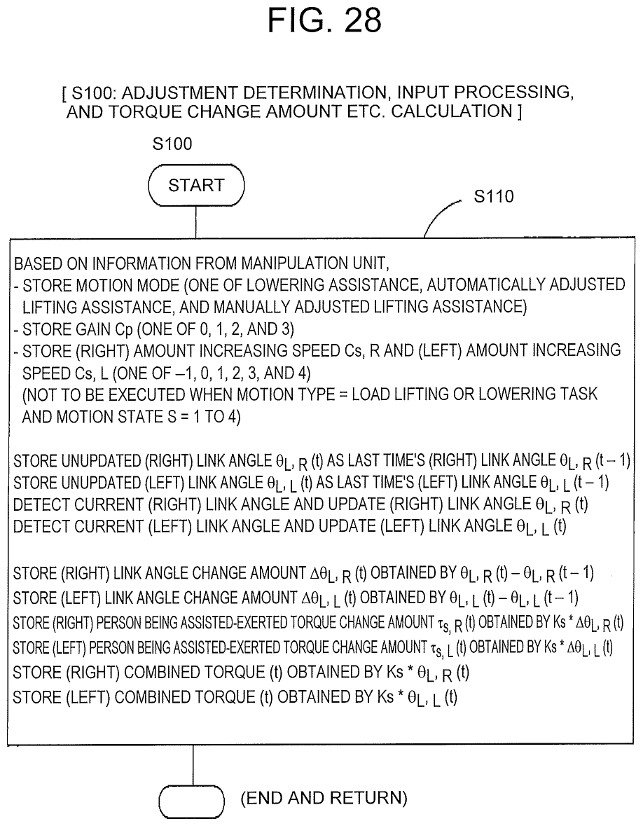

In step S010, the controller 61 executes a process S100 (see FIG. 28) and moves to step S020. The process S100 corresponds to the adjustment determination block B10, the input processing block B20, and the torque change amount etc. calculation block B30 shown in FIG. 26 and to the adjustment determination unit 61A, the input processing unit 61B, and the torque change amount etc. calculation unit 61C shown in FIG. 24. Details of the process S100 will be described later.

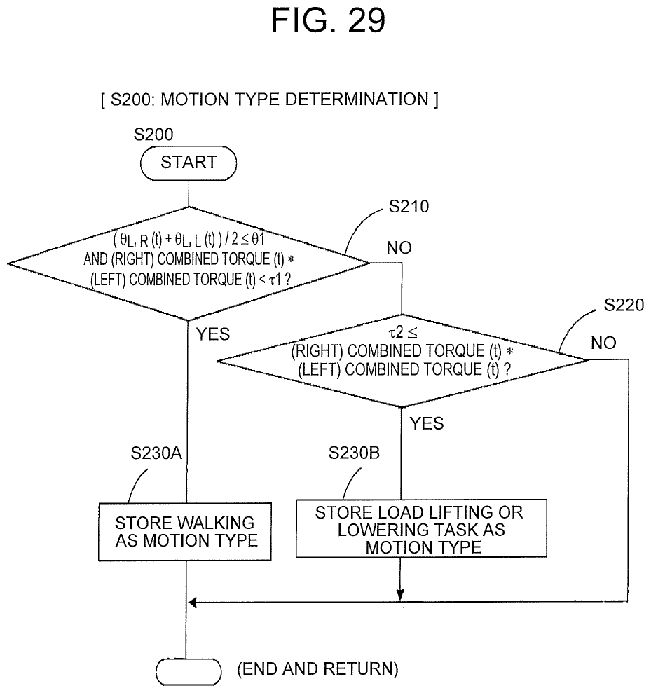

In step S020, the controller 61 executes a process S200 (see FIG. 29) and moves to step S030. The process S200 corresponds to the motion type determination block B40 shown in FIG. 26 and the motion type determination unit 61D shown in FIG. 24. Details of the process S200 will be described later.

In step S030, the controller 61 determines whether or not the motion type determined in step S020 is a load lifting or lowering task, and moves to step S035 if the motion type is a load lifting or lowering task (Yes) and moves to step S050 if not (No).

When the controller 61 moves to step S035, the controller 61 determines whether or not the motion mode (the motion mode from the manipulation unit) in step S010 is the lowering assistance, and moves to step S040R if the motion mode is the lowering assistance (Yes) and moves to step S045 if not (No). The processes in steps S030 and S035 correspond to the selection block B54 shown in FIG. 26 and the selection unit 61E shown in FIG. 24.

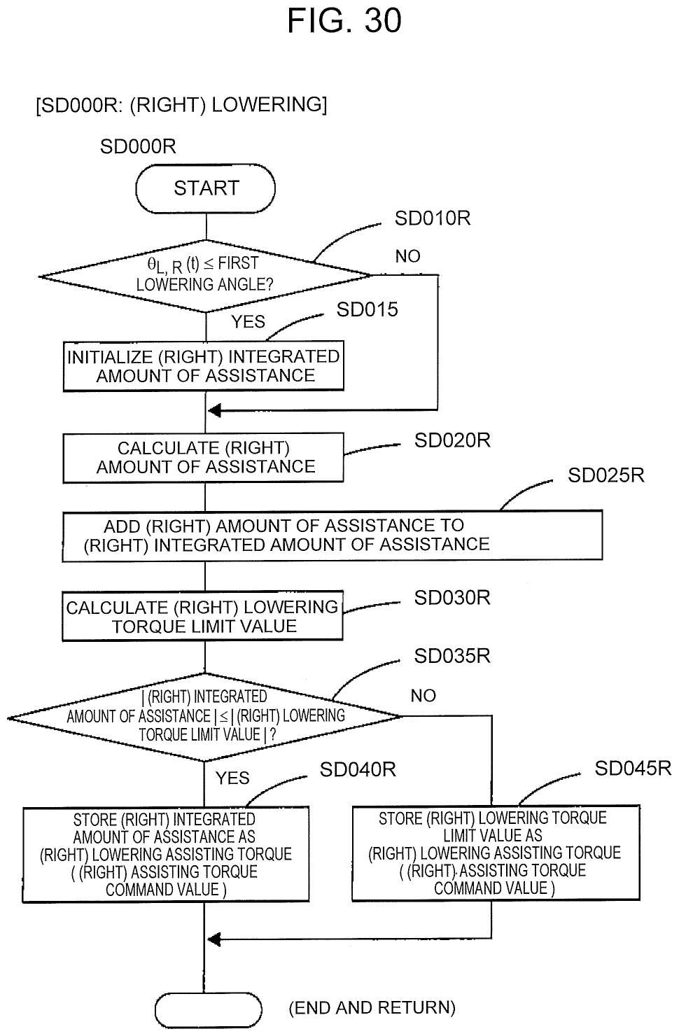

When the controller 61 moves to step S040R, the controller 61 executes a process SD000R (see FIG. 30) and moves to step S040L. The process SD000R is a process of obtaining a control command value for the (right) actuator unit 4R during a lowering motion, and corresponds to the lowering assisting torque calculation block B51 shown in FIG. 26 and the lowering assisting torque calculation unit 61F shown in FIG. 24. Details of the process SD000R will be described later.

In step S040L, the controller 61 executes a process SD000L (not shown) and moves to step S060R. The process SD000L is a process of obtaining a control command value for the (left) actuator unit 4L during a lowering motion, and corresponds to the lowering assisting torque calculation block B51 shown in FIG. 26 and the lowering assisting torque calculation unit 61F shown in FIG. 24. As the process SD000L is similar to SD000R, a detailed description thereof will be omitted.

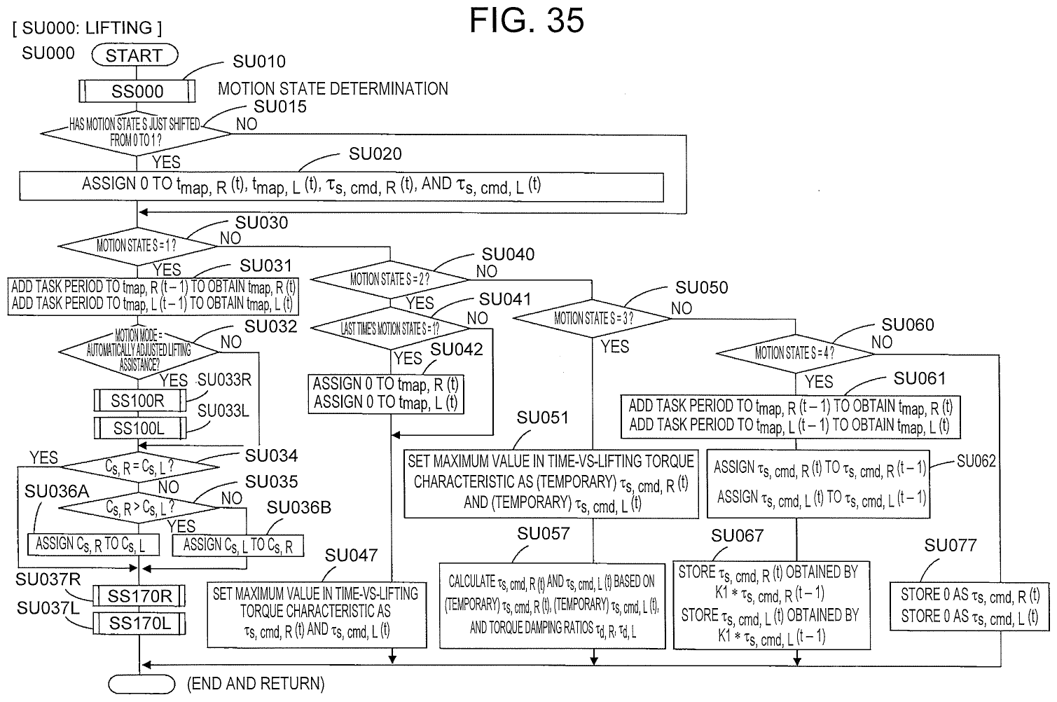

When the controller 61 moves to step S045, the controller 61 executes a process SU000 (see FIG. 35) and moves to step S060R. The process SU000 is a process of obtaining control command values for the (right) actuator unit 4R and the (left) actuator unit 4L during a lifting motion, and corresponds to the lifting assisting torque calculation block B52 shown in FIG. 26 and the lifting assisting torque calculation unit 61G shown in FIG. 24. Details of the process SU000 will be described later.

When the controller 61 moves to step S050, the controller 61 executes a process SW000 (not shown) and moves to step S060R. The process SW000 is a process of obtaining control command values for the (right) actuator unit 4R and the (left) actuator unit 4L during a walking motion, and corresponds to the walking assisting torque calculation block B53 shown in FIG. 26 and the walking assisting torque calculation unit 61H shown in FIG. 24. A detailed description of the process SW000 will be omitted.

In step S060R, the controller 61 performs feedback control on the (right) electric motor based on a (right) assisting torque command value obtained by the process SD000R or SU0000 or SW000, and moves to step S060L.

In step S060L, the controller 61 performs feedback control on the (left) electric motor based on a (left) assisting torque command value obtained by the process SD000L or SU000 or SW000, and ends the process. The processes in steps S060R and S060L correspond to the control command value calculation block B60 shown in FIG. 26 and the control command value calculation unit 61I shown in FIG. 24.

Next, the process S100 in step S010 shown in FIG. 27 will be described in detail by using FIG. 28. In the process S100, the controller 61 stores as the motion mode one of the lowering assistance, the automatically adjusted lifting assistance, and the manually adjusted lifting assistance, based on the information from the manipulation unit (see “Controller: Motion Mode” in FIG. 25). Further, the controller 61 stores one of 0, 1, 2, and 3 as the gain Cₚ based on the information from the manipulation unit (see “Controller: Gain” in FIG. 25). Except “when motion type=load lifting or lowering task and motion state S=1 to 4,” the controller 61 stores one of −1, 0, 1, 2, 3, and 4 as the (right) amount increasing speed Cₛ, _{R} and the (left) amount increasing speed Cₛ, _{L} based on the information from the manipulation unit (see “Controller: Amount Increasing Speed” in FIG. 25). This process corresponds to the adjustment determination block B10 shown in FIG. 26 and the adjustment determination unit 61A shown in FIG. 24.

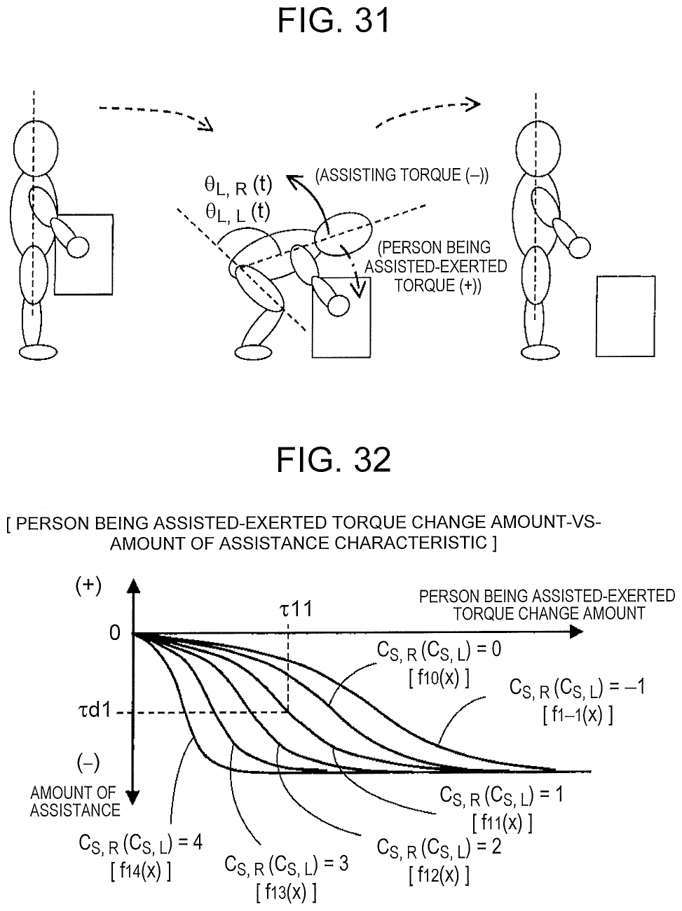

The controller 61 stores an unupdated (right) link angle θ_{L}, _{R} (t) as a last time's (right) link angle θ_{L}, _{R}(t−1), and stores an unupdated (left) link angle θ_{L}, _{L} (t) as a last time's (left) link angle θ_{L}, _{L} (t−1). Further, the controller 61 detects the current (right) link angle by using the output link turning angle detection means 43RS (an example of the “angle detection part”; see FIG. 19 and FIG. 20) of the (right) actuator unit, and stores the detected (right) link angle as the (right) link angle θ_{L}, _{R} (t) (updates the (right) link angle θ_{L}, _{R} (t) with the detected (right) link angle). Similarly, the controller 61 detects the current (left) link angle by using the output link turning angle detection means (an example of the “angle detection part”) of the (left) actuator unit, and stores the detected (left) link angle as the (left) link angle θ_{L}, _{L} (t) (updates the (left) link angle θ_{L}, _{L} (t) with the detected (left) link angle). This process corresponds to the input processing block B20 shown in FIG. 26 and the input processing unit 61B shown in FIG. 24. The (right) link angle θ_{L}, _{R} (t) is a (right) forward leaning angle of the hip relative to the thigh (see FIG. 31), and the (left) link angle θ_{L}, _{L} (t) is a (left) forward leaning angle of the hip relative to the thigh (see FIG. 31).

The controller 61 stores a (right) link angle change amount Δθ_{L}, _{R} (t) obtained by the following Formula 1, and stores a (left) link angle change amount Δθ_{L}, _{L} (t) obtained by the following Formula 2. Each of the (right) link angle change amount Δθ_{L}, _{R} (t) and the (left) link angle change amount Δθ_{L}, _{L} (t) corresponds to the “angular velocity-related amount.” The output link turning angle detection means 43RS is an example of the “torque detection part.”

(Right) link angle change amount Δθ_{L},_{R}=(right)link angle θ_{L},_{R}−(right)link angle θ_{L},_{R}(t−1) (Formula 1)

(Left) link angle change amount Δθ_{L},_{L}(t)=(left)link angle θ_{L},_{L}(t)−(left)link angle θ_{L},_{L}(t−1) (Formula 2)

The controller 61 stores a (right) person being assisted-exerted torque change amount τ_{S}, _{R} (t) obtained by the following Formula 3, and stores a (left) person being assisted-exerted torque change amount τ_{S}, _{L} (t) obtained by the following Formula 4. The symbol Ks represents the spring constant of the spiral spring 45R.

(Right) person being assisted-exerted torque change amount τ_{S},_{R}(t)=Ks*Δθ_{L},_{R}(t) (Formula 3)

(Left) person being assisted-exerted torque change amount τ_{S},_{L}(t)=Ks*Δθ_{L},_{L}(t) (Formula 4)

The controller 61 stores a (right) combined torque (t) obtained by the following Formula 5, and stores a (left) combined torque (t) obtained by the following Formula 6. This process corresponds to the torque change amount etc. calculation block B30 shown in FIG. 26 and the torque change amount etc. calculation unit 61C shown in FIG. 24.

(Right) combined torque (t)=Ks*Δθ_{L},_{R}(t) (Formula 5)

(Left) combined torque (t)=Ks*Δθ_{L},_{L}(t) (Formula 6)

Next, the process S200 in step S020 shown in FIG. 27 will be described in detail by using FIG. 29. In the process S200, the controller 61 determines the type of a motion of the person being assisted. The motion types to be determined include “walking” and “load lifting or lowering.” Walking is a walking motion of the person being assisted, and load lifting or lowering is a motion of the person being assisted lifting a heavy object or putting down a heavy object that the person being assisted is holding. The process S200 corresponds to the motion type determination block B40 shown in FIG. 26 and the motion type determination unit 61D shown in FIG. 24.

In the process S200, the controller 61 moves to step S210. In step S210, the controller 61 determines whether or not [(right) link angle θ_{L}, _{R} (t)+(left) link angle θ_{L}, _{L} (t)]/2 is equal to or smaller than a first motion determining angle θ1 and (right) combined torque (t)*(left) combined torque (t) is smaller than a first motion determining torque τ1. The controller 61 moves to step S230A if [(right) link angle θ_{L}, _{R} (t)+(left) link angle θ_{L}, _{L} (t)]/2 is equal to or smaller than the first motion determining angle θ1 and (right) combined torque (t)*(left) combined torque (t) is smaller than the first motion determining torque τ1 (Yes), and moves to step S220 if not (No).

When the controller 61 moves to step S220, the controller 61 determines whether or not (right) combined torque (t)*(left) combined torque (t) is equal to or larger than a second motion determining torque τ2, and moves to step S230B if (right) combined torque (t)*(left) combined torque (t) is equal to or larger than the second motion determining torque τ2 (Yes), and ends the process S200 and returns (moves to step S030 in FIG. 27) if not (No).

When the controller 61 moves to step S230A, the controller 61 stores “walking” as the motion type, and ends the process S200 and returns (moves to step S030 in FIG. 27).

When the controller 61 moves to step S230B, the controller 61 stores “load lifting or lowering task” as the motion type, and ends the process S200 and returns (moves to step S030 in FIG. 27).

Next, the process SD000R in step S040R shown in FIG. 27 will be described in detail by using FIG. 30. In the process SD000R, the controller 61 calculates a (right) lowering assisting torque to be generated by the assist device to assist the person being assisted in performing a lowering task. For the process SD000R, the procedure of the process of calculating the (right) lowering assisting torque to be generated by the (right) actuator unit 4R (see FIG. 1) is shown. The procedure of the process SD000L (see FIG. 27) of calculating a (left) lowering assisting torque to be generated by the (left) actuator unit 4L (see FIG. 1) is similar and therefore the description thereof will be omitted. As shown in FIG. 31, in a lowering task in which the person being assisted puts down a load that the person being assisted is holding, the (right) link angle θ_{L}, _{R} (t) and the (left) link angle θ_{L}, _{L} (t) are forward leaning angles of the hips relative to the thighs. The lowering assisting torque that assists the person being assisted in performing a task in a lowering direction (the direction of “person being assisted-exerted torque” in FIG. 31) is generated in a lifting direction relative to the person being assisted (the direction of “assisting torque” in FIG. 31). In the following description, the sign of a torque in the lifting direction and the sign of a torque in the lowering direction will be written as “−” (negative) and “+” (positive), respectively.

In the process SD000R, the controller 61 moves to step SD010R. In step SD010R, the controller 61 determines whether or not the (right) link angle θ_{L}, _{R} (t) is equal to or smaller than a first lowering angle θd1, and moves to step SD015R if the (right) link angle θ_{L}, _{R} (t) is equal to or smaller than the first lowering angle θd1 (Yes) and moves to step SD020R if not (No). For example, the first lowering angle θd1 is a forward leaning angle of about 10[°], and when θ_{L}, _{R} (t)≤θd1, the controller 61 determines that lowering has started or ended.

When the controller 61 moves to step SD015R, the controller 61 initializes (resets to zero) a (right) integrated amount of assistance and moves to step SD020R.

When the controller 61 moves to step SD020R, the controller 61 calculates a (right) amount of assistance based on the (right) amount increasing speed C_{R}, the (right) person being assisted-exerted torque change amount τ_{S}, _{R} (t), and a person being assisted-exerted torque change amount-vs-amount of assistance characteristic (FIG. 32), and moves to step SD025R. As shown in FIG. 32, for example, when the (right) amount increasing speed Cₛ, _{R}=1 and the (right) person being assisted-exerted torque change amount τ_{S}, _{R}=τ11, the controller 61 uses the characteristic f11 (x) of Cₛ, _{R}=1 and thereby obtains all corresponding to τ11 as the (right) amount of assistance.

In step SD025R, the controller 61 adds the (right) amount of assistance obtained in step SD020R to the (right) integrated amount of assistance (i.e., integrates the obtained (right) amount of assistance), and moves to step SD030R.

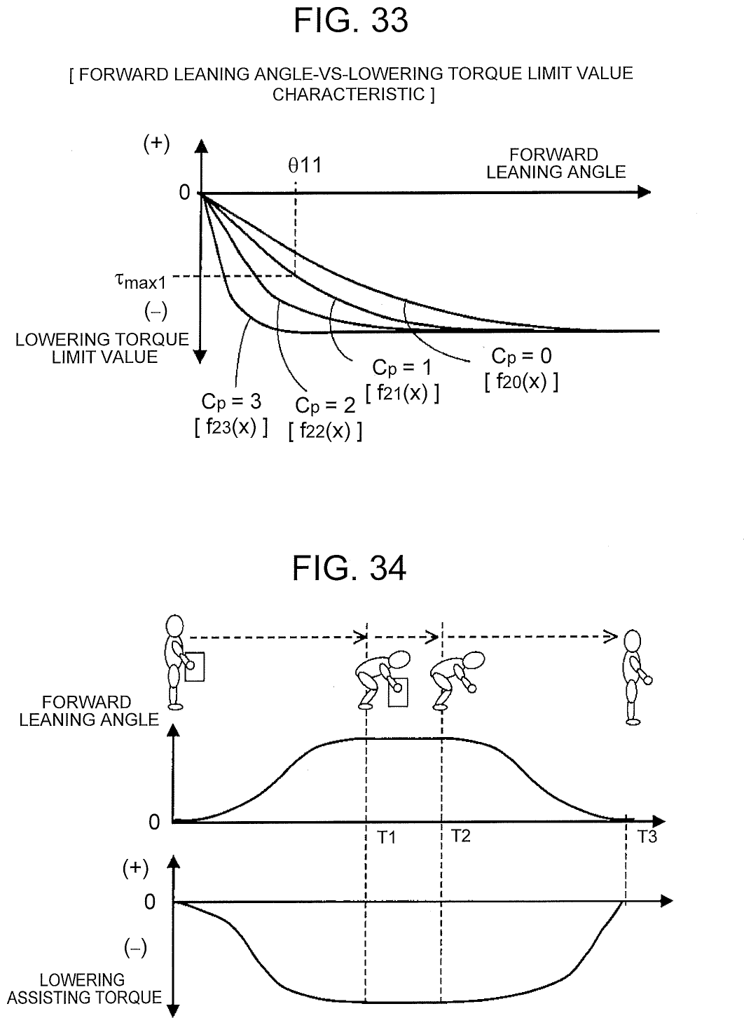

In step SD030R, the controller 61 calculates a (right) lowering torque limit value based on the gain Cₚ, the (right) link angle (forward leaning angle) θ_{L}, _{R} (t), and a forward leaning angle-vs-lowering torque limit value characteristic (see FIG. 33), and moves to step SD035R. As shown in FIG. 33, for example, when the gain Cₚ=1 and the (right) link angle (forward leaning angle) θ_{L}, _{R}=011, the controller 61 uses the characteristic f21 (x) of Cₚ=1 and thereby obtains τmax1 corresponding to θ11 as the (right) lowering torque limit value.

In step SD035R, the controller 61 determines whether or not |(right) integrated amount of assistance| is equal to or smaller than |(right) lowering torque limit value|, and moves to step SD040R if |(right) integrated amount of assistance| is equal to or smaller than |(right) lowering torque limit value| (Yes) and moves to step SD045R if not (No).

When the controller 61 moves to step SD040R, the controller 61 stores the (right) integrated amount of assistance as the (right) lowering assisting torque (i.e., a (right) assisting torque command value τₛ, _{cmd}, _{R} (t)), and ends the process and returns (moves to step S060R in FIG. 27).

When the controller 61 moves to step SD045R, the controller 61 stores the (right) lowering torque limit value as the (right) lowering assisting torque (i.e., the (right) assisting torque command value τₛ, _{cmd}, _{R} (t)), and ends the process and returns (moves to step S060R in FIG. 27).

By steps SD035R, SD040R, and SD045R, the controller 61 sets |(right) integrated amount of assistance| or |(right) lowering torque limit value|, whichever is the smaller, as the (right) lowering assisting torque.

FIG. 34 shows how the lowering assisting torque is set so as to correspond to the forward leaning angle during a lowering task by the above process. The example shown in FIG. 34 shows a case where the person being assisted holds a load in an upright standing posture at time 0, completes lowering of the load at time T1 while gradually increasing the forward leaning angle, maintains a forward leaning posture until time T2, and returns to the upright standing posture while gradually reducing the forward leaning angle. In this case, the lowering assisting torque in the lifting direction (toward the − (negative) side in FIG. 34) is as shown in FIG. 34, and thus the assist device can appropriately provide assistance in the lowering task by reducing the load on the hips of the person being assisted.

When the person being assisted stops a forward leaning motion and the forward leaning angle stops changing (Δθ_{L}, _{R} (t)=0, Δθ_{L}, _{L} (t)=0); (in the example of FIG. 34, from time T1 to time T2), or when the person being assisted is performing an upright standing motion in which the person being assisted gradually reduces the forward leaning angle from the forward leaning posture (in the example of FIG. 34, from time T2 to time T3), the person being assisted-exerted torque change amount is zero or directed in the opposite direction, so that the amount of assistance obtained from the person being assisted-exerted torque change amount-vs-amount of assistance characteristic (see FIG. 32) becomes zero. In this case, the controller 61 stops updating and retains the integrated amount of assistance, and obtains the (right) lowering assisting torque ((right) assisting torque command value) based on the retained integrated amount of assistance and the lowering torque limit value.

Next, the process SU000 in step S045 shown in FIG. 27 will be described in detail by using FIG. 35. In the process SU000, the controller 61 calculates a lifting assisting torque to be generated by the assist device to assist the person being assisted in performing a lifting task. In the lifting task in which the person being assisted lifts a load, the (right) link angle θ_{L}, _{R} (t) and the (left) link angle θ_{L}, _{L} (t) (see FIG. 31) are forward leaning angles of the hips relative to the thighs. The lifting assisting torque that assists the person being assisted in performing a task in the lifting direction is generated in the lifting direction relative to the person being assisted (the direction of “assisting torque” in FIG. 31). In the following description, the sign of a torque in the lifting direction and the sign of a torque in the lowering direction will be written as “−” (negative) and “+” (positive), respectively.

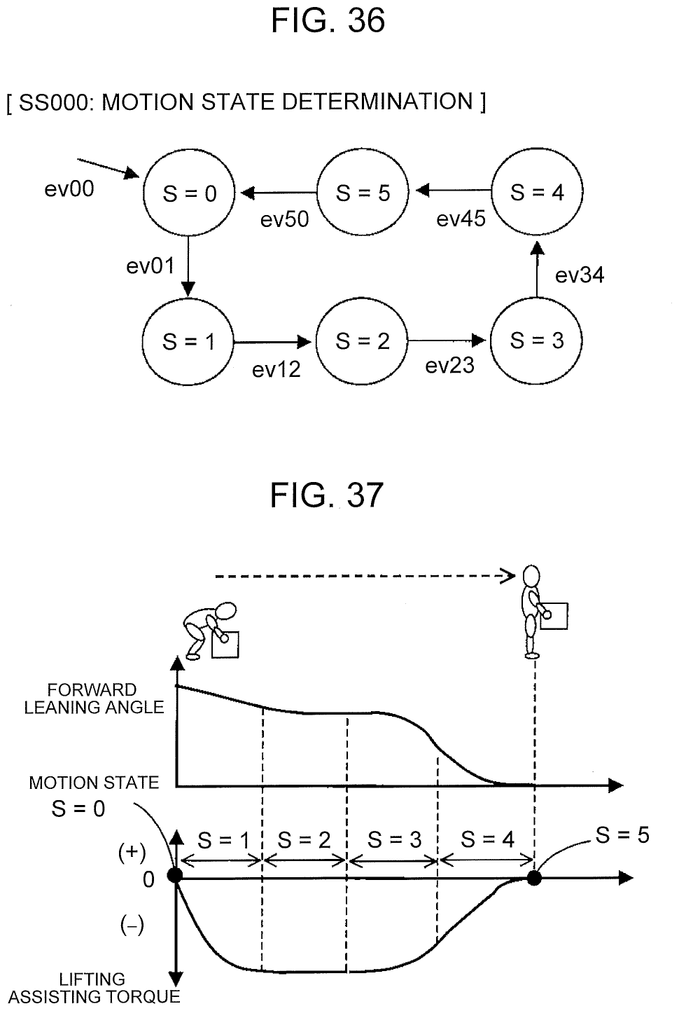

In the process SU000, the controller 61 moves to step SU010. In step SU010, the controller 61 executes the process SS000 (see FIG. 36) and moves to step SU015. As shown in the state shift chart of FIG. 36, the process SS000 is a process of determining the current motion state S, with the entire lifting motion from the start to the end of lifting divided into six motion states S of 0 to 5. Details of this process will be described later.

In step SU015, the controller 61 determines whether or not the motion state S has just shifted from 0 to 1, and moves to step SU020 if the motion state S has just shifted from 0 to 1 (Yes) and moves to step SU030 if not (No).

When the controller 61 moves to step SU020, the controller 61 assigns 0 to a (right) virtual elapsed time tₘₐₚ, _{R} (t) and a (left) virtual elapsed time tₘₐₚ, _{L} (t), and assigns 0 to the (right) lifting assisting torque ((right) assisting torque command value τₛ, _{cmd}, _{R} (t)) and the (left) lifting assisting torque ((left) assisting torque command value τₛ, _{cmd}, _{L} (t)). Then, the controller 61 moves to step SU030.

When the controller 61 moves to step SU030, the controller 61 determines whether or not the motion state S determined in step SU020 is 1, and moves to step SU031 if the motion state S is 1 (Yes) and moves to step SU040 if not (No).

When the controller 61 moves to step SU031, the controller 61 adds a task period (e.g., 2 [ms] in the case where the process shown in FIG. 27 is started every 2 [ms]) to the (right) virtual elapsed time tₘₐₚ, _{R} (t), and adds the task period to the (left) virtual elapsed time tₘₐₚ, _{L} (t), and moves to step SU032. Each of the (right) virtual elapsed time tₘₐₚ, _{R} (t) and the (left) virtual elapsed time tₘₐₚ, _{L} (t) represents a (virtual) time that has elapsed since the motion state S became 1.

In step SU032, the controller 61 determines whether or not the motion mode is the “automatically adjusted lifting assistance,” and moves to step SU033R if the motion mode is the “automatically adjusted lifting assistance” (Yes) and moves to step SU034 if not (No).

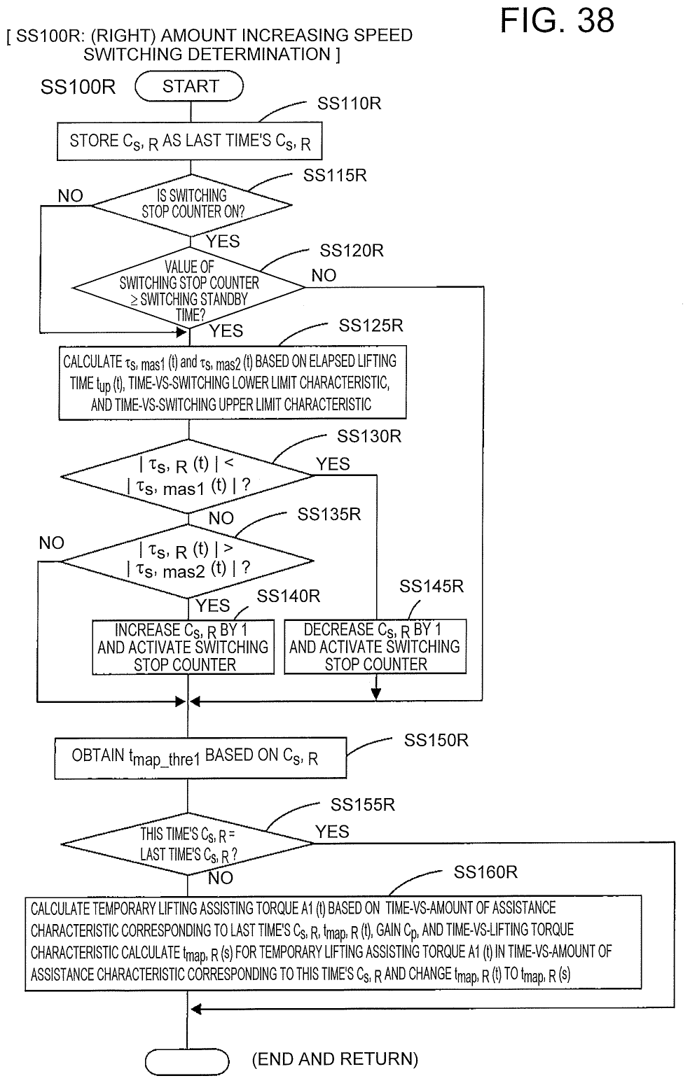

When the controller 61 moves to step SU033R, the controller 61 executes a process SS100R (see FIG. 38) and moves to step SU033L. The process SS100R (see FIG. 38) is a process of changing or maintaining the (right) amount increasing speed Cₛ, _{R} and the (right) virtual elapsed time tₘₐₚ, _{R} (t). A process SS100L is a similar process of changing or maintaining the (left) amount increasing speed Cₛ, _{L} and the (left) virtual elapsed time tₘₐₚ, _{L} (t), and therefore the description thereof will be omitted. In step SU033L, the controller 61 executes the process SS100L and moves to step SU034. Details of the process SS100R will be described later.

In step SU034, the controller 61 determines whether or not the (right) amount increasing speed Cₛ, _{R} and the (left) amount increasing speed Cₛ, _{L} are equal, and moves to step SU037R if the (right) amount increasing speed Cₛ, _{R} and the (left) amount increasing speed Cₛ, _{L} are equal (Yes) and moves to step SU035 if not (No).

When the controller 61 moves to step SU035, the controller 61 determines whether or not the (right) amount increasing speed Cₛ, _{R} is higher than the (left) amount increasing speed Cₛ, _{L}, and moves to step SU036A if the (right) amount increasing speed Cₛ, _{R} is higher than the (left) amount increasing speed Cₛ, _{L} (Yes) and moves to step SU036B if not (No).

When the controller 61 moves to step SU036A, the controller 61 assigns the (right) amount increasing speed Cₛ, _{R} to the (left) amount increasing speed Cₛ, _{L} and moves to step SU037R.

When the controller 61 moves to step SU036B, the controller 61 assigns the (left) amount increasing speed Cₛ, _{L} to the (right) amount increasing speed Cₛ, _{R} and moves to step SU037R.

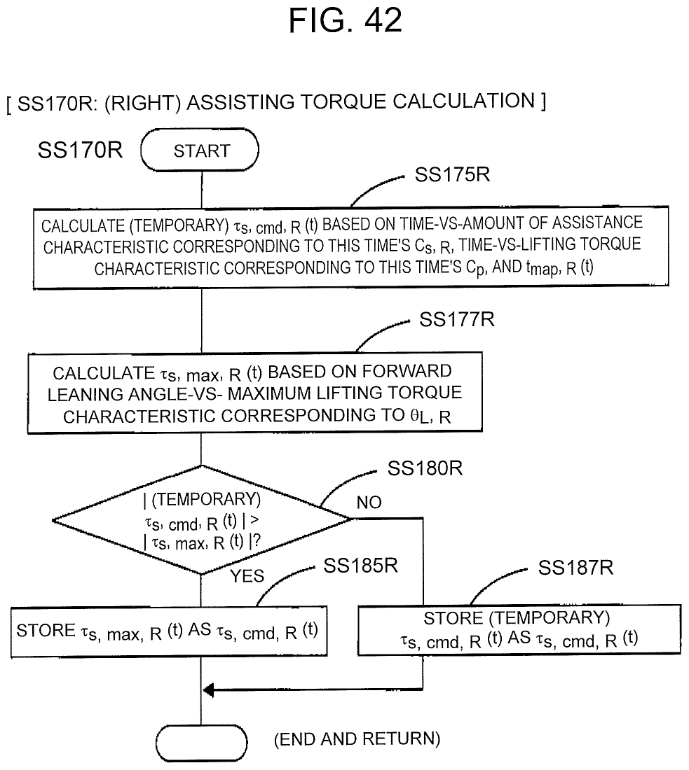

When the controller 61 moves to step SU037R, the controller 61 executes a process SS170R (see FIG. 42) and moves to step SU037L. The process SS170R (see FIG. 42) is a process of obtaining the (right) lifting assisting torque ((right) assisting torque command value τₛ, _{cmd}, _{R} (t)) in the case where the motion state S=1. A process SS170L is a similar process of obtaining the (left) lifting assisting torque ((left) assisting torque command value τₛ, _{cmd}, _{L} (t)) in the case where the motion state S=1, and therefore the description thereof will be omitted. In step SU037L, the controller 61 executes the process SS170L, and ends the process and returns (moves to step S060R in FIG. 27). Details of the process SS170R will be described later.

When the controller 61 moves to step SU040, the controller 61 determines whether or not the motion state S determined in step SU020 is 2, and moves to step SU041 if the motion state S is 2 (Yes) and moves to step SU050 if not (No).

When the controller 61 moves to step SU041, the controller 61 determines whether or not the (last time's) motion state S is 1, and moves to step SU042 if the (last time's) motion state S is 1 (Yes) and moves to step SU047 if not (No).

When the controller 61 moves to step SU042, the controller 61 assigns 0 to the (right) virtual elapsed time tₘₐₚ, _{R} (t) and the (left) virtual elapsed time tₘₐₚ, _{L} (t) and moves to step SU047. The process in step SU042 is a process executed when the motion state S has shifted from 1 to 2.

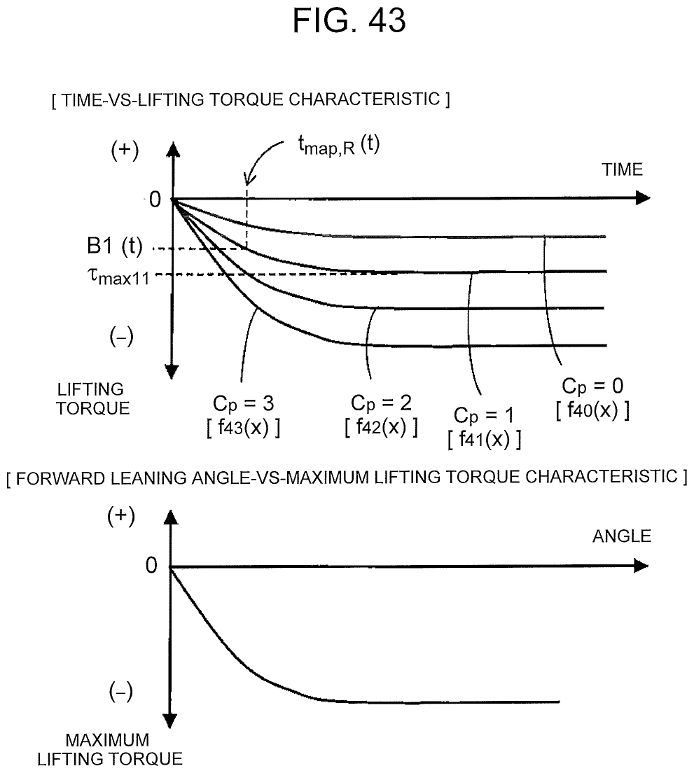

When the controller 61 moves to step SU047, the controller 61 obtains a |maximum value| corresponding to the gain Cₚ based on the gain Cₚ and a time-vs-lifting torque characteristic (see FIG. 43), and assigns the obtained maximum value to the (right) lifting assisting torque ((right) assisting torque command value τₛ, _{cmd}, _{R} (t)) and the (left) lifting assisting torque ((left) assisting torque command value τₛ, _{cmd}, _{L} (t)), and ends the process and returns (moves to step S060R in FIG. 27). For example, in the case where the gain Cₚ=1, the controller 61 uses the characteristic f41 (x) of Cₚ=1 in FIG. 43 and thereby obtains τmax11 that is the maximum value of |f41 (x)|, as the maximum value. As shown in FIG. 43, the time-vs-lifting torque characteristic (one of the “reference lifting characteristics”) is prepared according to the gain Cₚ, and the controller 61 changes the reference lifting characteristic according to the gain Cₚ.

When the controller 61 moves to step SU050, the controller 61 determines whether or not the motion state S determined in step SU020 is 3, and moves to step SU051 if the motion state S is 3 (Yes) and moves to step SU060 if not (No).

When the controller 61 moves to step SU051, the controller 61 obtains a maximum value corresponding to the gain Cₚ based on the gain Cₚ and the time-vs-lifting torque characteristic (see FIG. 43), and assigns the obtained maximum value to a (temporary) (right) lifting assisting torque ((temporary) τₛ, _{cmd}, _{R} (t)) and a (temporary) (left) lifting assisting torque ((temporary) τₛ, _{cmd}, _{L} (t)), and moves to step SU057. For example, in the case where the gain Cₚ=1, the controller 61 uses the characteristic f41 (x) of Cₚ=1 in FIG. 43 and thereby obtains τmax11 that is the maximum value of |f41 (x)|, as the maximum value.

In step SU057, the controller 61 obtains a (right) torque damping ratio τ_{d}, _{R} based on the gain Cₚ, the (right) person being assisted-exerted torque change amount τ_{S}, _{R} (t), and an assistance ratio-vs-torque damping ratio characteristic (see FIG. 45). Similarly, the controller 61 obtains a (left) torque damping ratio τ_{d}, _{L} based on the gain Cₚ, the (left) person being assisted-exerted torque change amount τ_{S}, _{L} (t), and the assistance ratio-vs-torque damping ratio characteristic (see FIG. 45). The controller 61 stores the (right) assisting torque command value τₛ, _{cmd}, _{R} (t) obtained by the following Formula 7, and stores the (left) assisting torque command value τₛ, _{and}, _{L} (t) obtained by the following Formula 8. Then, the controller 61 ends the process and returns (moves to step S060R in FIG. 27).

(Right) assisting torque command value tₛ,_{cmd},_{R}(t)=(temporary) τₛ,_{cmd},_{R}*(right) torque damping ratio τ_{d},_{R} (Formula 7)

(Left) assisting torque command value τₛ,_{cmd},_{L}(t)=(temporary)τₛ,_{and},_{L}(t)*(left) torque damping ratio τ_{d},_{L} (Formula 8)

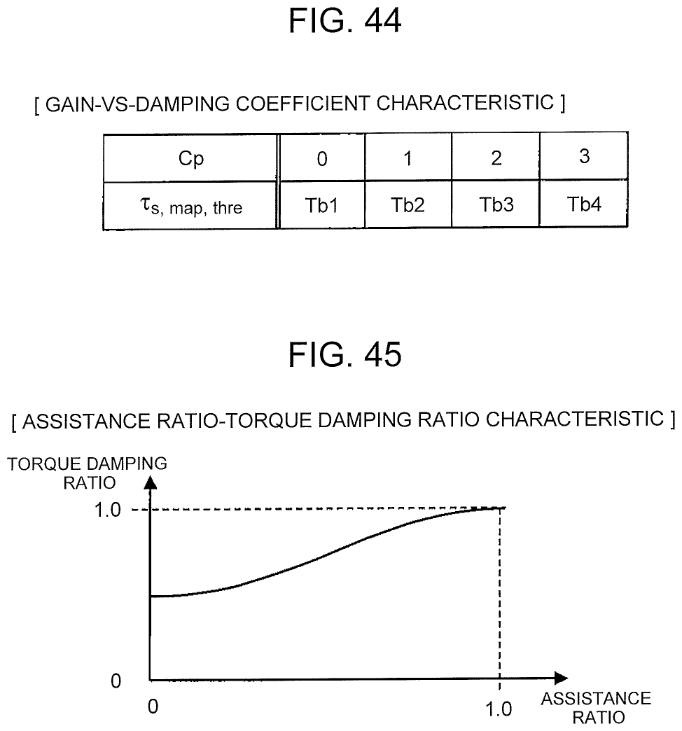

For example, in the case where the gain Cₚ=1, the controller 61 obtains a damping coefficient τₛ, ₘₐₚ, ₜᵢₜᵣₑ=Tb2 based on the gain-vs-damping coefficient characteristic shown in FIG. 44. Then, the controller 61 calculates a (right) assistance ratio by the following Formula 9 and calculates a (left) assistance ratio by the following Formula 10.

(Right) assistance ratio=[τ_{S},ₘₐₚ,ₜₕᵣₑ−(right) person being assisted-exerted torque change amount τ_{S},_{R}(t)]/τₛ,ₘₐₚ,ₜₕᵣₑ (Formula 9)

(Left) assistance ratio=[τₛ,ₘₐₚ,ₜᵢₜᵣₑ−(left) person being assisted-exerted torque change amount τₛ,_{L}(t)]/τₛ,ₘₐₚ,ₜₕᵣₑ (Formula 10)

The controller 61 obtains the (right) torque damping ratio τ_{d}, _{R} based on the (right) assistance ratio and the assistance ratio-vs-torque damping ratio characteristic (see FIG. 45), and obtains the (left) torque damping ratio τ_{d}, _{L} based on the (left) assistance ratio and the assistance ratio-vs-torque damping ratio characteristic (see FIG. 45). Then, the controller 61 stores the result of (temporary) τₛ, _{cmd}, _{R} (t)*(right) torque damping ratio τ_{d}, _{R} as the (right) lifting assisting torque ((right) assisting torque command value τₛ, _{cmd}, _{R} (t)), and stores the result of (temporary) τₛ, _{cmd}, _{L} (t)*(left) torque damping ratio τ_{d}, _{L} as the (left) lifting assisting torque ((left) assisting torque command value τₛ, _{cmd}, _{L} (t)).

When the controller 61 moves to step SU060, the controller 61 determines whether or not the motion state S determined in step SU020 is 4, and moves to step SU061 if the motion state S is 4 (Yes) and moves to step SU077 if not (No).

When the controller 61 moves to step SU061, the controller 61 adds a task period (e.g., 2 [ms] in the case where the process shown in FIG. 27 is started every 2 [ms]) to the (right) virtual elapsed time tₘₐₚ, _{R} (t), and adds the task period to the (left) virtual elapsed time tₘₐₚ, _{L} (t), and moves to step SU062. Each of the (right) virtual elapsed time tₘₐₚ, _{R} (t) and the (left) virtual elapsed time tₘₐₚ, _{L} (t) represents a (virtual) time that has elapsed since the motion state S became 4.

In step SU062, the controller 61 assigns the current τₛ, _{cmd}, _{R} (t) to the (last time's) τₛ, _{cmd}, _{R} (t−1) and assigns the current τₛ, _{cmd}, _{L} (t) to the (last time's) τₛ, _{cmd}, _{L} (t−1), and moves to step SU067.

In step SU067, the controller 61 stores the (right) assisting torque command value τₛ, _{cmd}, _{R} (t) obtained by the following Formula 11, and stores the (left) assisting torque command value τₛ, _{and}, _{L} (t) obtained by the following Formula 12. A damping coefficient K1 is a preset coefficient, which is set to 0.9, for example. Then, the controller 61 ends the process and returns (moves to step S060R in FIG. 27).

(Right) assisting torque command value τₛ,_{cmd},_{R}(t)=K1*(last time's)τₛ,_{cmd},_{R}(t−1) (Formula 11)

(Left) assisting torque command value τₛ,_{cmd},_{L}=K1*(last time's)τₛ,_{cmd},_{L}(t−1) (Formula 12)

When the controller 61 moves to step SU077, the controller 61 stores the (right) assisting torque command value τₛ, _{and}, _{R} (t) obtained by the following Formula 13, and stores the (left) assisting torque command value τₛ, _{cmd}, _{L} (t) obtained by the following Formula 14. Then, the controller 61 ends the process and returns (moves to step S060R in FIG. 27).

(Right) assisting torque command value τₛ,_{cmd},_{R}=0 (Formula 13)

(Left) assisting torque command value τₛ,_{cmd},_{L}=0 (Formula 14)

As has been described above, during a lifting task, the controller 61 shifts the motion state S sequentially from 0 to 5 according to the lifting state, and obtains the (right) lifting assisting torque ((right) assisting torque command value τₛ, _{cmd}, _{R} (t)) and the (left) lifting assisting torque ((left) assisting torque command value τₛ, _{cmd}, _{L} (t)) in accordance with the preset calculation methods corresponding to the respective motion states S.

Next, the process SS000 in step SU020 shown in FIG. 35 will be described in detail by using FIG. 36. In the process SS000, the controller 61 determines the motion state S=0 to 5 according to the lifting state during a lifting task of the person being assisted. An overview of the motion state S is as shown in FIG. 37: 0 represents a motion state S at a point in time when the person being assisted starts to lean forward from the upright standing posture (the posture in which the person being assisted has stopped leaning forward in the preceding task) and starts a lifting motion; 1 represents a motion state S to which the motion state shifts after the lifting motion has started; 2 represents a motion state S where the person being assisted is performing a load lifting motion; 3 and 4 represent motion states S where the person being assisted gradually reduces the forward leaning angle; and 5 represents a motion state S where the person being assisted has completed lifting of the load and assumed the upright standing posture. The motion state S is set according to the lifting state including at least one of the (right) virtual elapsed time tₘₐₚ, _{R} (t), the (left) virtual elapsed time tₘₐₚ, _{L} (t), the (right) link angle (forward leaning angle) θ_{L}, _{R} (t), the (left) link angle (forward leaning angle) θ_{L}, _{L} (t), the (right) person being assisted-exerted torque change amount τₛ, _{R} (t), and the (left) person being assisted-exerted torque change amount τ_{S}, _{L} (t).

In the following, the procedure of determining the motion state S will be described by using the state shift chart shown in FIG. 36. As shown in FIG. 36, the controller 61 determines that the motion state S is 0 by an event ev00 that lifting has started. Whether or not lifting has started can be determined based on the (right) link angle θ_{L}, _{R} (t), the (left) link angle θ_{L}, _{L} (t), the (right) link angle change amount Δθ_{L}, _{R} (t), the (left) link angle change amount Δθ_{L}, _{L} (t), the (right) person being assisted-exerted torque change amount τ_{S}, _{R} (t), the (left) person being assisted-exerted torque change amount τ_{S}, _{L} (t), etc. In the case where the motion state S=0, the controller 61 shifts the motion state S from 0 to 1 upon detecting an event ev01. The event ev01 is “always,” and therefore, as shown in step SU015 in FIG. 35, the controller 61 unconditionally shifts the motion state S to 1 after shifting the motion state S to 0.

In the case where the motion state S=1, the controller 61 shifts the motion state S from 1 to 2 upon detecting an event ev12. When the event ev12 is not detected, the controller 61 maintains the motion state S=1. For example, the event ev12 is detected as having occurred when the condition “(right) virtual elapsed time tₘₐₚ, _{R} (t)≥(right) tₘₐₚ, ₜₕᵣₑ₁” is met or the condition “(left) virtual elapsed time tₘₐₚ, _{L}≥(t)≥(left) tₘₐₚ, ₜₕᵣₑ₁” is met, or when either the (right) link angle (forward leaning angle) θ_{L}, _{R} (t) or the (left) link angle (forward leaning angle) θ_{L}, _{L} (t) becomes a forward leaning angle equivalent to that of near the end of the lifting task. The (right) tₘₐₚ, ₜₕᵣₑ₁ determined based on the (right) amount increasing speed Cₛ, _{R} and an amount increasing speed-vs-shift time characteristic (see FIG. 40), and the (left) tₘₐₚ, ₜₕᵣₑ₁ is determined based on the (left) amount increasing speed Cₛ, _{L} and the amount increasing speed-vs-shift time characteristic (see FIG. 40.)

In the case where the motion state S=2, the controller 61 shifts the motion state S from 2 to 3 upon detecting an event ev23. When the event ev23 is not detected, the controller 61 maintains the motion state S=2. For example, the event ev23 is detected as having occurred when the (right) person being assisted-exerted torque change amount τₛ, _{R} (t) or the (left) person being assisted-exerted torque change amount τ_{S}, _{L} (t) becomes a relatively small amount equivalent to that of near the end of the lifting task, or when the (right) link angle (forward leaning angle) θ_{L}, _{R} (t) or the (left) link angle (forward leaning angle) θ_{L}, _{L} (t) becomes a forward leaning angle equivalent to that of near the end of the lifting task.

In the case where the motion state S=3, the controller 61 shifts the motion state S from 3 to 4 upon detecting an event ev34. When the event ev34 is not detected, the controller 61 maintains the motion state S=3. For example, the event ev34 is detected as having occurred when the condition “(right) person being assisted-exerted torque change amount τ_{S}, _{R} (t)≥tₛ, ₘₐₚ, ₜₕᵣₑ” is met or the condition “(left) person being assisted-exerted torque change amount τ_{S}, _{L} (t)≥τₛ, ₘₐₚ, ₜᵢₜᵣₑ” is met, or when the (right) link angle (forward leaning angle) θ_{L}, _{R} (t) or the (left) link angle (forward leaning angle) θ_{L}, _{L} (t) becomes a forward leaning angle equivalent to that of near the end of the lifting task. The damping coefficient τₛ, ₘₐₚ, ₜₕᵣₑ is determined based on the gain Cₚ and the gain-vs-damping coefficient characteristic (see FIG. 44).

In the case where the motion state S=4, the controller 61 shifts the motion state S from 4 to 5 upon detecting an event ev45. When the event ev45 is not detected, the controller 61 maintains the motion state S=4. For example, the event ev45 is detected as having occurred when the condition “(right) virtual elapsed time tₘₐₚ, _{R} (t)≥state determining time t41 (e.g., about 0.15 [sec])” is met or the condition “(left) virtual elapsed time tₘₐₚ, _{L} (t)≥state determining time t41 (e.g., about 0.15 [sec])” is met.

In the case where the motion state S=5, the controller 61 shifts the motion state S from 5 to 0 upon detecting an event ev50. When the event ev50 is not detected, the controller 61 maintains the motion state S=5. The event ev50 is a start of the lifting task, and the motion state S returns to 0 upon completion of the lifting task.

Next, the process SS100R in step SU033R shown in FIG. 35 will be described in detail by using FIG. 38. In the process SS100R, the controller 61 automatically switches the (right) amount increasing speed Cₛ, _{R} to an appropriate value from −1 to 4 according to the lifting motion of the person being assisted. For the process SS100R, the procedure of the process of automatically switching the (right) amount increasing speed Cₛ, _{R} is shown. The procedure of the process SS100L (see FIG. 35) of automatically switching the (left) amount increasing speed Cₛ, _{L} is similar and therefore the description thereof will be omitted.

In the process SS100R, the controller 61 moves to step SS110R. In step SS110R, the controller 61 stores the current (right) amount increasing speed Cₛ, _{R} as the last time's Cₛ, _{R} and moves to step SS115R.

In step SS115R, the controller 61 determines whether or not a switching stop counter is on, and moves to step SS120R if the switching stop counter is on (Yes) and moves to step SS125R if not (No). The switching stop counter is a counter that is activated when the (right) amount increasing speed Cₛ, _{R} is switched (changed) in steps SS140R and SS145R.

When the controller 61 moves to step SS120R, the controller 61 determines whether or not the value of the switching stop counter is equal to or larger than a switching standby time, and moves to step SS125R if the value of the switching stop counter is equal to or larger than the switching standby time (Yes) and moves to step SS150R if not (No).

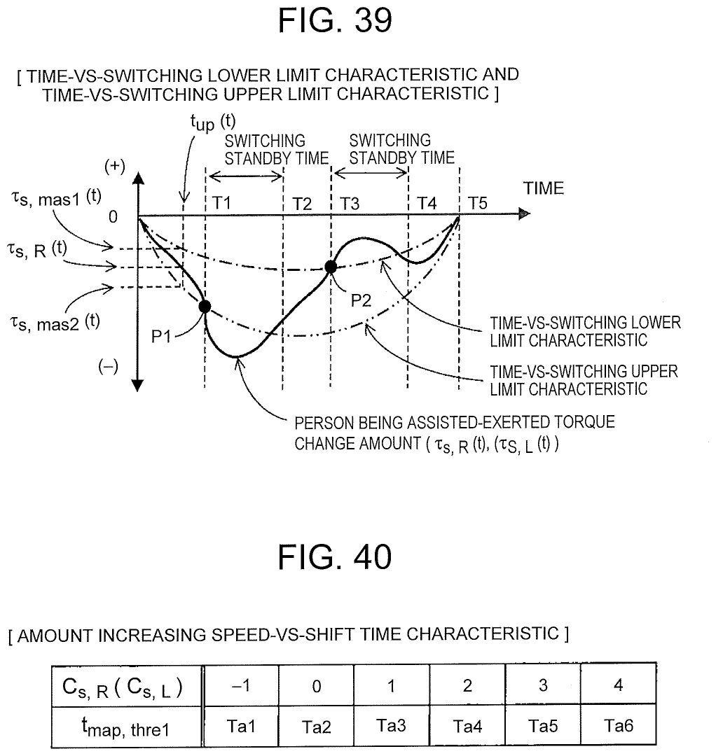

When the controller 61 moves to step SS125R, the controller 61 obtains a switching lower limit τₛ, ₘₐₛ₁ (t) corresponding to the current elapsed lifting time tᵤₚ (t) based on an elapsed lifting time tᵤₚ, (t) and a time-vs-switching lower limit characteristic (see FIG. 39). Further, the controller 61 obtains a switching upper limit τₛ, ₘₐₛ₂ (t) corresponding to the current elapsed lifting time tᵤₚ (t) based on the current (right) amount increasing speed Cₛ, _{R}, the elapsed lifting time tᵤₚ (t), and the time-vs-switching upper limit characteristic (see FIG. 39). The elapsed lifting time tᵤₚ (t) is a time that has elapsed since lifting started (the motion state S shifted from 0 to 1). Then, the controller 61 moves to step SS130R. The example shown in FIG. 39 is an example in which the condition “|(right) person being assisted-exerted torque change amount τ_{S}, _{R}(t)|>|switching upper limit τₛ, ₘₐₛ₂ (t)|” is met at time T1 (at the point P1) and the condition “|(right) person being assisted-exerted torque change amount τ_{S}, _{R}(t)|<|switching upper limit τₛ, ₘₐₛ₂ (t)|” is met at time T3 (at the point P2).

In step SS130R, the controller 61 determines whether or not |(right) person being assisted-exerted torque change amount τ_{S}, _{R} (t)| is smaller than |switching lower limit τₛ, ₘₐₛ₁ (t)|, and moves to step SS145R if |(right) person being assisted-exerted torque change amount τ_{S}, _{R} (t)| is smaller than |switching lower limit τₛ, ₘₐₛ₁ (t)| (Yes) and moves to step SS135R if not (No).

When the controller 61 moves to step SS135R, the controller 61 determines whether or not |(right) person being assisted-exerted torque change amount τₛ, _{R} (t)| is larger than |switching upper limit τₛ, ₘₐₛ₂ (t)|, and moves to step SS140R if |(right) person being assisted-exerted torque change amount τₛ, _{R} (t)| is larger than |switching upper limit τₛ, ₘₐₛ₂ (t)| (Yes) and moves to step SS150R if not (No).

When the controller 61 moves to step SS140R, the controller 61 increases the value of the (right) amount increasing speed Cₛ, _{R} by 1 (with a guard “maximum value=4”) and activates the switching stop counter, and moves to step SS150R.

When the controller 61 moves to step SS145R, the controller 61 decreases the value of the (right) amount increasing speed Cₛ, _{R} by 1 (with a guard “minimum value=−1”) and activates the switching stop counter, and moves to step SS150R.

When the controller 61 moves to step SS150R, the controller 61 obtains (right) tₘₐₚ, ₜₕᵣₑ₁ based on the (right) amount increasing speed Cₛ, _{R} and the amount increasing speed-vs-shift time characteristic (see FIG. 40), and moves to step SS155R. The (right) tₘₐₚ, ₜₕᵣₑ₁ is used for determination of the motion state (determination of a shift of the motion state from 1 to 2) etc.

In step SS155R, the controller 61 determines whether or not this time's (current) (right) amount increasing speed Cₛ, _{R} is equal to the last time's Cₛ, _{R} (see step SS110R), and ends the process and returns (returns to step SU033L in FIG. 35) if this time's (right) amount increasing speed Cₛ, _{R} is equal to the last time's Cₛ, _{R} (Yes) and moves to step SS160R if not (No).

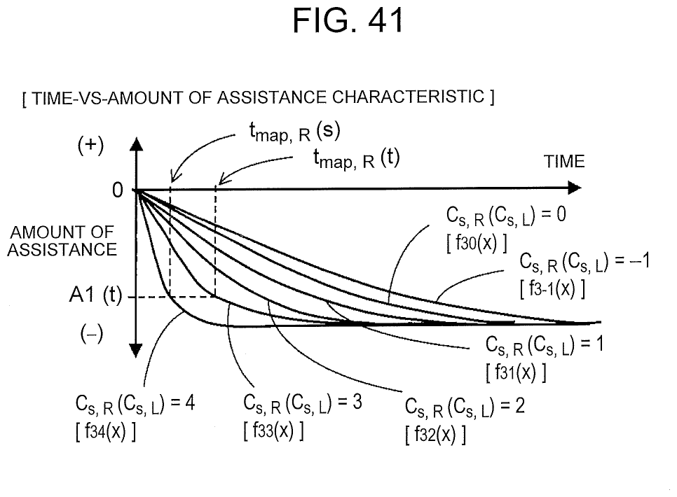

When the controller 61 moves to step SS160R, the controller 61 calculates a temporary lifting assisting torque A1 (t) based on the last time's Cₛ, _{R}, the (right) virtual elapsed time tₘₐₚ, _{R} (t), the time-vs-amount of assistance characteristic (see FIG. 41), the gain Cₚ, and the time-vs-lifting torque characteristic (see FIG. 43). For example, in the case where the last time's Cₛ, _{R}=3, as shown in FIG. 41, the controller 61 calculates the temporary lifting assisting torque A1 (t) from f33 (x) corresponding to Cₛ, _{R}=3 and the (right) virtual elapsed time tₘₐₚ, _{R} (t). As shown in FIG. 41, the time-vs-amount of assistance characteristic (one of the “reference lifting characteristics”) is prepared according to the (right) amount increasing speed Cₛ, _{R} and the (left) amount increasing speed Cₛ, _{L}, and the controller 61 changes the reference lifting characteristic according to the (right) amount increasing speed Cₛ, _{R} and the (left) amount increasing speed Cₛ, _{L}.

The controller 61 calculates a torque difference-reducing virtual elapsed time tₘₐₚ, _{R} (s) corresponding to the temporary lifting assisting torque A1 (t) based on the this time's (current) (right) amount increasing speed Cₛ, _{R}, the time-vs-amount of assistance characteristic (see FIG. 41), the gain Cₚ, and the time-vs-lifting torque characteristic (see FIG. 43), and assigns the calculated torque difference-reducing virtual elapsed time tₘₐₚ, _{R} (s) to the (right) virtual elapsed time tₘₐₚ, _{R} (t) (rewrites the (right) virtual elapsed time tₘₐₚ, _{R} (t) with the calculated torque difference-reducing virtual elapsed time tₘₐₚ, _{R} (s)). For example, in the case where this time's (current) (right) amount increasing speed Cₛ, _{R}=4, as shown in FIG. 41, the controller 61 calculates the torque difference-reducing virtual elapsed time tₘₐₚ, _{R} (s) from f34 (x) corresponding to Cₛ, _{R}=4 and the temporary lifting assisting torque A1 (t), and assigns the torque difference-reducing virtual elapsed time tₘₐₚ, _{R} (s) to the (right) virtual elapsed time tₘₐₚ, _{R} (t). Then, the controller 61 ends the process and returns (returns to step SU033L in FIG. 35). The rewriting of the (right) virtual elapsed time tₘₐₚ, _{R} (t) corresponds to the “on-switching torque difference-reducing correction” of, when the motion state S has shifted to a predetermined motion state S (in this case, when the motion state S has shifted to 1), reducing the difference between the lifting assisting torque (temporary lifting assisting torque A1(t)) obtained based on the selected reference lifting characteristic (the time-vs-amount of assistance characteristic corresponding to the last time's (right) amount increasing speed Cₛ, _{R} (see FIG. 41)) and the lifting assisting torque obtained based on the currently selected reference lifting characteristic (the time-vs-amount of assistance characteristic corresponding to this time's (current) (right) amount increasing speed Cₛ, _{R} (see FIG. 41)).

In the above description, the time-vs-amount of assistance characteristic (see FIG. 41), and the time-vs-lifting torque characteristic and the forward leaning angle-vs-maximum lifting torque characteristic (see FIG. 43) correspond to the “reference lifting characteristics” having a set lifting assisting torque that is a torque in the lifting direction. The controller 61 selects an appropriate reference lifting characteristic and obtains the lifting assisting torque based on the selected reference lifting characteristic, and drives the actuator unit based on the assisting torque that is the obtained lifting assisting torque.

Next, the process SS170R in step SU037R shown in FIG. 35 will be described in detail by using FIG. 42. In the process SS170R, the controller 61 obtains the (right) lifting assisting torque ((right) assisting torque command value τₛ, _{cmd}, _{R} (t)). For the process SS170R, the procedure of the process of obtaining the (right) lifting assisting torque ((right) assisting torque command value τₛ, _{cmd}, _{R} (0) is shown. The procedure of the process of obtaining the (left) lifting assisting torque ((left) assisting torque command value τₛ, _{cmd}, _{L} (t)) is similar and therefore the description thereof will be omitted.

In the process SS170R, the controller 61 moves to step SS175R. In step SS175R, the controller 61 calculates (temporary) τₛ, _{cmd}, _{R} (t) based on this time's (current) (right) amount increasing speed Cₛ, _{R}, the (right) virtual elapsed time tₘₐₚ, _{R} (t), the gain Cₚ, the time-vs-amount of assistance characteristic (see FIG. 41), and the time-vs-lifting torque characteristic (see FIG. 43), and moves to step SS177R. For example, in the case where this time's (current) (right) amount increasing speed Cₛ, _{R}=3, as shown in FIG. 41, the controller 61 stores as (temporary) τₛ, _{cmd}, _{R} (t) the assisting torque A1 (t) obtained from f33 (x) corresponding to Cₛ, _{R}=3 and the (right) virtual elapsed time tₘₐₚ, _{R} (t).

In step SS177R, the controller 61 calculates a (right) torque upper limit value τₛ, ₘₐₓ, _{R} (t) based on the forward leaning angle and the forward leaning angle-vs-maximum lifting torque characteristic (see FIG. 43), and moves to step SS180R. For example, the controller 61 stores as the (right) torque upper limit value τₛ, ₘₐₓ, _{R} (t) a maximum lifting torque B1 (t) obtained from the forward leaning angle-vs-maximum lifting torque characteristic shown in FIG. 43 and the (right) link angle (forward leaning angle) θ_{L}, _{R} (t). The torque value of the lifting torque is limited by the “forward leaning angle-vs-maximum lifting torque characteristic” so as not to become too large when the forward leaning angle is small.

In step SS180R, the controller 61 determines whether or not |(temporary) τₛ, _{cmd}, _{R} (t)| is larger than |(right) torque upper limit value τₛ, ₘₐₓ, _{R} (t)|, and moves to step SS185R if |(temporary) τₛ, _{cmd}, _{R} (t)| is larger than |(right) torque upper limit value τₛ, ₘₐₓ, _{R} (t)| (Yes) and moves to step SS187R if not (No).

When the controller 61 moves to step SS185R, the controller 61 stores the (right) torque upper limit value τₛ, ₘₐₓ, _{R} (t) as the (right) lifting assisting torque ((right) assisting torque command value τₛ, _{cmd}, _{R} (t)), and ends the process and returns (returns to step SU037L in FIG. 35).

When the controller 61 moves to step SS187R, the controller 61 stores (temporary) τₛ, _{cord}, _{R} (t) as the (right) lifting assisting torque ((right) assisting torque command value τₛ, _{cmd}, _{R} (t)), and ends the process and returns (returns to step SU037L in FIG. 35).

Thus, the assist device 1 described in the embodiment has a simple configuration and can be easily worn by a person being assisted. The assisting control for a lowering task and the assisting control for a lifting task are both simple, and the assist device 1 can appropriately provide assistance in a load lifting task and a load lowering task. Alternatively, the assist device 1 described in the embodiment may perform only either the assisting control for a lowering task or the assisting control for a lifting task.

Various changes, additions, and omissions can be made to the structure, configuration, shape, external appearance, processing procedure, etc. of the assist device of the present disclosure to such an extent as not to change the gist of the disclosure. For example, the processing procedure of the controller is not limited to the flowchart etc. described in the above embodiment. While the example of using the spiral spring 45R (see FIG. 19) has been described in the embodiment, a torsion spring (a torsion bar or a torsion bar spring) may be used instead of a spiral spring.

In the embodiment of the assist device 1 described above, the example of using an adjuster or a buckle as the belt retaining member for retaining a belt in a fastened state has been described. The example of connecting and disconnecting the belt etc. by a buckle has been described, but a belt retaining member other than a buckle may also be used to connect and disconnect the belt etc. While in the above example the belt is passed through the adjuster such that the tensioned belt does not loosen, a belt retaining member other than an adjuster may also be used. Alternatively, a belt retaining member having the functions of both an adjuster and a buckle may also be used.

In the above embodiment, the example of the manipulation unit R1 having both the gain upward and downward manipulation parts R1BU, R1BD and the amount increasing speed upward and downward manipulation parts R1CU, R1CD has been described. However, the manipulation unit R1 may be configured to have at least either the gain upward and downward manipulation parts R1BU, R1BD or the amount increasing speed upward and downward manipulation parts R1CU, R1CD.

In the embodiment of the assist device 1 described above, the example in which the motion mode, the gain, the amount increasing speed, etc. can be changed through the manipulation unit R1 has been described. Alternatively, the controller 61 may be provided with the communication means 64 (that communicates in a wireless or wired manner; see FIG. 24) such that such changes can be made through communication from a smartphone etc. The controller 61 may include the communication means 64 (that communicates in a wireless or wired manner; see FIG. 24), and the controller 61 may collect various pieces of data and send the collected pieces of data to an analysis system at predetermined timing (e.g., at all times, at regular time intervals, or at the end of an assisting motion). Examples of the data to be collected include person-being-assisted information and assistance information. For example, the person-being-assisted information is information on the person being assisted, including the person being assisted-exerted torque and the posture of the person being assisted. For example, the assistance information is information on inputs into and outputs from the right and left actuator units, including the assisting torque, the rotation angle of the electric motor (actuator) (the actual motor shaft angle θ_{rM} in FIG. 24), the output link turning angle (the actual link angle θ_{L} in FIG. 24), the motion mode, the gain, and the amount increasing speed. The analysis system is a system that is prepared separately from the assist device, and that is incorporated in a device, for example, an external personal computer, a server, a programmable logic controller (PLC), or a computerized numerical control (CNC) device, connected via a network (LAN). The analysis system may analyze (calculate) optimal setting values (optimal values of the gain, the amount increasing speed, etc.) specific to the assist device 1 (i.e., specific to the person being assisted), and analysis information including the optimal setting values that are an analysis result (calculation result) may be sent to the controller 61 (communication means 64) of the assist device 1. By analyzing the motion of the person being assisted, an assisting force, etc. by the analysis system, it is possible to output an optimal assisting torque with the type of task (repetitive or not, the height of lifting, etc.) and the capacity of the person being assisted taken into account. Based on the analysis information (e.g., the gain and the amount increasing speed) received from the analysis system, the right and left actuator units adjust their own operations (e.g., change the gain and the amount increasing speed to those that have been received).

What is claimed is:

1. An assist device comprising:
body gear worn at least around hips of a person being assisted;
an actuator unit attached to the body gear and worn on thighs of the person, the actuator unit being configured to generate an assisting torque that assists the person in moving his or her thighs relative to the hips or moving his or her hips relative to the thighs;
an angle detection part configured to detect a forward leaning angle of the hips relative to the thighs of the person; and
a controller configured to control the actuator unit,
the controller being configured to execute at least one of first control and second control,
the first control involving, during a lifting task, obtaining a lifting assisting torque based on the forward leaning angle detected by using the angle detection part, an angular velocity-related amount based on a change in the forward leaning angle, and a reference lifting characteristic, and then driving the actuator unit based on the assisting torque that is the lifting assisting torque, the lifting task being a task in which the person lifts a load that the person is holding in a forward leaning posture, while gradually reducing the forward leaning angle, and
the second control involving, during a lowering task, obtaining a lowering assisting torque that is a torque in a lifting direction, based on the forward leaning angle detected by using the angle detection part and the angular velocity-related amount based on the change in the forward leaning angle, and then driving the actuator unit based on the assisting torque that is the obtained lowering assisting torque, the lowering task being a task in which the person lowers a load that the person is holding, in a lowering direction opposite from the lifting direction while gradually increasing the forward leaning angle.

2. The assist device according to claim 1, further comprising a torque detection part configured to detect a person exerted torque change amount that is an amount of change in a person exerted torque that is a torque input from the person into the actuator unit as the person moves his or her thighs relative to the hips or moves his or her hips relative to the thighs by himself or herself, wherein:
the controller is configured to, when the person is performing a forward leaning motion of gradually increasing the forward leaning angle from an upright standing posture during the lowering task, detect the person exerted torque change amount at predetermined time intervals based on the angular velocity-related amount detected by using the torque detection part; and
the controller is configured to obtain an amount of assistance according to the person exerted torque change amount, and to obtain the lowering assisting torque based on an integrated amount of assistance obtained by integrating the amount of assistance.

3. The assist device according to claim 2, wherein
the controller is configured to, when a predetermined state arises during the lowering task, stop updating and retain the integrated amount of assistance, and then obtain the lowering assisting torque based on the retained integrated amount of assistance,
the predetermined state being either a state where the forward leaning angle has stopped changing as the person has stopped the forward leaning motion or a state where the person is performing an upright standing motion of gradually reducing the forward leaning angle from the forward leaning posture.

4. The assist device according to claim 1, further comprising a storage unit, wherein:
the storage unit is configured to store a forward leaning angle-vs-lowering torque limit value characteristic having a torque limit value set according to the forward leaning angle;
the controller is configured to, during the lowering task, obtain the torque limit value based on the forward leaning angle and the forward leaning angle-vs-lowering torque limit value characteristic that is stored in the storage unit; and
the controller is configured to use an integrated amount of assistance or the torque limit value, whichever is the smaller, as the lowering assisting torque.

5. The assist device according to claim 1, further comprising a manipulation unit provided with at least one of a gain changing part and an amount increasing speed changing part, the gain changing part being configured to allow the person to change a gain in the lowering assisting torque, the amount increasing speed changing part being configured to allow the person to change a speed with which an amount of the lowering assisting torque is increased, the manipulation unit being separate from the body gear and the actuator unit, wherein:
when the manipulation unit is provided with the gain changing part, the controller is configured to, during the lowering task, increase and decrease at least either a gain used to obtain the lowering assisting torque, or a torque limit value according to the forward leaning angle, based on an input from the person into the gain changing part; and
when the manipulation unit is provided with the amount increasing speed changing part, the controller is configured to change the speed with which the amount of the lowering assisting torque is increased, based on an input from the person into the amount increasing speed changing part.

6. The assist device according to claim 1, further comprising a manipulation unit provided with a motion switching part, the motion switching part being configured to switch between lowering assistance of assisting the person in performing a motion during the lowering task and lifting assistance of assisting the person in performing a motion during the lifting task, wherein:
the manipulation unit is separate from the body gear and the actuator unit; and
the controller is configured to, when the motion switching part represents the lowering assistance during the lowering task, obtain the lowering assisting torque and then drive the actuator unit based on the assisting torque that is the lowering assisting torque.

7. The assist device according to claim 1, further comprising a storage unit, wherein:
the storage unit is configured to store a plurality of reference lifting characteristics having a set lifting assisting torque that is a torque in a lifting direction; and
the controller is configured to, during the lifting task, select an applicable reference lifting characteristic from the reference lifting characteristics stored in the storage unit, and obtain the lifting assisting torque based on the forward leaning angle detected by using the angle detection part, the angular velocity-related amount based on the change in the forward leaning angle, and the selected reference lifting characteristic, and then drive the actuator unit based on the assisting torque that is the lifting assisting torque.

8. The assist device according to claim 7, further comprising a torque detection part configured to detect a person exerted torque change amount that is an amount of change in a person exerted torque that is a torque input from the person into the actuator unit as the person moves his or her thighs relative to the hips or moves his or her hips relative to the thighs by himself or herself, wherein:
each of the reference lifting characteristics has a plurality of motion states set according to a lifting state, the lifting state including at least one of a virtual elapsed time based on a time that has elapsed since the person started to lift a load, the forward leaning angle, and the person exerted torque change amount; and
the controller is configured to, during the lifting task, shift each of the motion states in the reference lifting characteristics based on the lifting state, and obtain the lifting assisting torque by a calculation method that is preset for each of the motion states.

9. The assist device according to claim 8, wherein
the controller is configured to, during the lifting task, when the reference lifting characteristic currently selected is different from the reference lifting characteristic selected last time or when the motion state has shifted to a predetermined motion state among the motion states, make an on-switching torque difference-reducing correction of reducing a predetermined difference, the predetermined difference being a difference between the lifting assisting torque obtained based on the reference lifting characteristic selected last time and the lifting assisting torque obtained based on the reference lifting characteristic currently selected.

10. The assist device according to claim 9, wherein:
in the predetermined motion state in each of the reference lifting characteristics, the lifting assisting torque is set according to the virtual elapsed time; and
the controller is configured to, during the lifting task, when the motion state has shifted to the predetermined motion state or when the reference lifting characteristic currently selected is different from the reference lifting characteristic selected last time, obtain a temporary lifting assisting torque based on a current virtual elapsed time and the reference lifting characteristic selected last time, and obtain a torque difference-reducing virtual elapsed time that is the virtual elapsed time corresponding to the temporary lifting assisting torque in the reference lifting characteristic currently selected, and then change the current virtual elapsed time to the torque difference-reducing virtual elapsed time.
